(19) United States
(12) Patent Application Publication
(10) Pub. No.: US 2021/0062160 A1
(43) Pub. Date: Mar. 4, 2021

(54) THERAPEUTIC AND IMMUNOMODULATORY BACTERIOPHAGE FORMULATIONS AND METHODS FOR MAKING AND USING THEM

(71) Applicants: San Diego State University (SDSU) Foundation, dba San Diego State University Research Foundation, San Diego, CA (US); THE BRIGHAM AND WOMEN'S HOSPITAL, INC., Boston, MA (US)

(72) Inventors: Forest ROHWER, San Diego, CA (US); Jeremy J. BARR, San Diego, CA (US); Richard S. BLUMBERG, Boston, MA (US); Kristi D. BAKER, Boston, MA (US)

(21) Appl. No.: 16/476,800

(22) PCT Filed: Jan. 9, 2018
(86) PCT No.: PCT/US18/12983

§ 371 Date: Jul. 9, 2019

Related U.S. Application Data
(60) Provisional application No. 62/444,269, filed on Jan. 9, 2017.

(51) Int. Cl. C12N 7/00; C12N 15/86

(52) U.S. Cl. CPC C12N 7/00; C12N 15/86; C12N 2795/00043; C12N 2795/00032; C12N 2795/00034; C12N 2795/00021

(57) ABSTRACT

In alternative embodiments, provided are compositions, products of manufacture and methods for treating, ameliorating and preventing infections, disorders and conditions in animals including: delivering a (i) bacteriophage (ii) prophage, the phagemid or phage-like particle, (iii) general transducing agent (GTA), or small, tailed bacteriophage-like particle, (iv) Metamorphosis Associated Contractile structure (MACs) or (v) phage-derived product into a tissue, or the blood stream or lymphatic system of the animal, e.g., the mammal; or delivering to a tissue or organ of the animal; or treating a bacterial or viral infection in the animal; generating an immune response in the animal; or treating a disease or condition in an individual in need thereof; or delivering a payload or a composition, e.g., in vivo, to the animal, or labelling, tagging or coating a cell in vivo in the animal.

### RELATED APPLICATIONS

This U.S. Utility Patent Application claims the benefit of priority under 35 U.S.C. § 119(e) of U.S. Provisional Application Ser. No. (USSN) 62/444,269, filed Jan. 9, 2017. The aforementioned application is expressly incorporated herein by reference in its entirety and for all purposes.

### STATEMENT AS TO FEDERALLY SPONSORED RESEARCH

This invention was made with government support under grant no. DK53056. The government has certain rights in the invention.

### TECHNICAL FIELD

This invention generally relates to medicine, infectious diseases, immunology, pharmacology and microbiology. In alternative embodiments, provided are compositions and methods for treating, ameliorating and preventing various infections, disorders and conditions in mammals, including: delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), into the blood stream or lymphatic system of an animal, e.g., a mammal, or delivering the bacteriophage, phagemid or phage-like particle, etc to a tissue or organ of the animal in vivo; treating, ameliorating and/or preventing a bacterial or viral infection in the animal in vivo, wherein the bacterial or viral infection in the animal is outside of the gut of the mammal, wherein optionally the bacterial or viral infection comprises a lung infection, or a secondary infection outside of the gut; generating an immune response in the animal by delivering the bacteriophage, phagemid or phage-like particle, etc into the blood stream or lymphatic system of the mammal, wherein optionally the immune response is a humoral (antibody) response, a cell-mediated immune response, or a tolerogenic immune (suppressing) response; treating, ameliorating and/or preventing a disease or condition in an individual in need thereof, wherein optionally the disease or condition comprises obesity, diabetes, autism, a cystic fibrosis, an inflammation outside of the gut; and/or delivering a payload or a composition in vivo to the animal, or labelling, tagging or coating a cell in vivo in the animal, comprising administering or applying to the animal in vivo, or to an individual in need thereof: a composition, a product of manufacture, a food, a drink, a nutraceutical, a formulation, a pharmaceutical or a pharmaceutical preparation comprising the bacteriophage, phagemid or phage-like particle, etc, which optionally comprise a payload, e.g., a drug, an effector nucleic acid, an immunogen, a label.

### BACKGROUND

Bacteriophages can freely and profusely penetrate our bodies and the bodies of other higher vertebrates (4, 5)(6, 7). Phages have been detected in the blood and serum of both symptomatic and asymptomatic humans (8-12). Dosing phages to mice via oral feeding and gastric lavage resulted in the rapid migration of phage into the blood stream that was both irregular but repeatable (6). Phage migration to the blood was rapidly followed by their permeation into all major organs of the body, including the lung, liver, kidney, spleen, urinary tract and even the brain, indicating their capacity to cross the blood-brain barrier (6, 13-16).

Within the human body the largest reservoir of phages is within the gut (17, 18). From here there are several possible routes by which gut phages could penetrate the body. The most rudimentarily proposed route of access is via a ‘leaky gut’, characterized by cellular damage and punctured vasculature at sites of inflammation, allowing phages to bypass confluent epithelial layers (19, 20). Other proposed mechanisms include; ‘trojan horse’ whereby phages infect a bacterium, which then enters or is engulfed by an epithelial cells (21-23), ‘phage display’ a process that requires homing ligands to be displayed onto viral coats for cellular recognition and receptor-mediated transcytosis (24-28), and the ‘free uptake’ of phage particles by eukaryotic cells via endocytosis (22, 29, 30). There is supporting and contrasting evidence for all of these mechanisms, suggesting that phages may access the body via diverse routes. Few attempts have been made to investigate whether phage transcytosis occurs naturally and, consequently the primary route that phages use to access the body has yet to be identified.

### SUMMARY

In alternative embodiments, provided are methods for:

delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into an animal (optionally a mammal or a human),
optionally the delivering is into a tissue, the blood stream or lymphatic system of the animal, wherein optionally the delivering is ex vivo or in vivo, and optionally the animal is a mammal or a human,

delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into a eukaryotic cell,
wherein optionally the delivering comprises entering or injecting into a subcellular compartment or an organelle of the eukaryotic cell, and optionally the eukaryotic cell subcellular compartment or organelle comprises or is a cytoplasm, an endosome, an exosome, a liposome, a nucleus, a nucleosome, a golgi, an endoplasmic reticulum (ER) or a mitochondria, and optionally the eukaryotic cell is in or derived from a mammal or a human,

treating, ameliorating and/or preventing a bacterial or viral infection in an animal in vivo, wherein optionally the bacterial or viral infection in the animal is inside or outside of the gut of the animal,
wherein optionally the bacterial or viral infection comprises a gut, muscle, lung, liver, kidney or blood (sepsis) infection, or a secondary infection inside or outside of the gut,
and optionally the animal is a mammal or a human,

generating or modulating an immune response in an animal by delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into the animal (optionally a mammal or a human),
optionally delivering into a tissue, the blood stream or lymphatic system of the animal, or into a cell of the animal,
wherein optionally the immune response is a humoral (antibody) response, a cell-mediated immune response, or a tolerogenic immune (suppressing) response, and optionally the modulating of the immune response decreases, ameliorates or inhibits inflammation or an autoimmune reaction in the animal,
and optionally the decreasing, ameliorating or inhibiting of inflammation or the autoimmune reaction in the animal treats, ameliorates, decreases the severity of or inhibits a disease or condition caused by an inflammation or an autoimmune reaction or a disease or condition causing an inflammation or autoimmune reaction,
and optionally the immune response is modulated by inclusion of, release from or display on the surface of (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), an immunogen or a tolerogen,

treating, ameliorating and/or preventing a disease or condition in an individual in need thereof,
wherein optionally the disease or condition comprises obesity, diabetes, autism, a cystic fibrosis, an inflammation outside or outside of the gut,
and optionally the individual is an animal, a mammal, or a human, and/or

delivering a payload or a composition in vivo to an animal, or labelling, tagging or coating a cell in vivo in an animal,
wherein optionally the payload is delivered into a eukaryotic cell (intracellular delivery of the payload), wherein optionally the payload comprises a small molecule or a nucleic acid,
and optionally the animal is a mammal or a human, the method comprising:

administering or applying: to the animal, optionally in vivo, or to the individual in need thereof; or, or administering or applying or inserting into or onto the eukaryotic cell:
(a) (i) the bacteriophage (“phage”) (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v); or,
(b) a composition, a product of manufacture, a food, a drink, a nutraceutical, a formulation, a pharmaceutical or a pharmaceutical preparation comprising: (i) the bacteriophage (“phage”) (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v),

wherein optionally the (i) the bacteriophage (“phage”) (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v) is: chemically or structurally modified, genetically engineered, or is a synthetic version or construct,

and optionally the (i) the bacteriophage (“phage”) (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v), comprises or has contained thereon or within a payload, wherein optionally the payload comprises a composition heterologous to (i) the bacteriophage (“phage”) (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product,

and optionally the heterologous composition is capable of treating, ameliorating and/or preventing a disease or condition in the individual in need thereof, or repairing a defect in the eukaryotic cell, or adding or modifying a function in the eukaryotic cell, or altering the genome of or a nucleic acid in the eukaryotic cell,

and optionally the (i) the bacteriophage (“phage”) (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v), has a size ranging from between about 1 nm and 1000 nm, or between about 100 and 500 nm, or between about 1 nm and 10 μm.

In alternative embodiments, the individual is a mammal or a human, and optionally the mammal is a human, a human infant, and optionally the animal is wildlife, livestock, beef, poultry, or a domesticated or a laboratory animal.

In alternative embodiments, an antacid or a buffer or buffering agent or a pharmaceutically acceptable excipient is administered before, during or after, or before and during, administration of the composition, product of manufacture, food, drink, nutraceutical, formulation, pharmaceutical or pharmaceutical preparation.

In alternative embodiments, a sufficient amount of antacid, buffer or buffering agent is administered (optionally before, during or after, or before and during, administration) to raise the pH of the stomach in the individual to between about 2.5 and 7, or between about 3 and 6.5, or to about 5.0, 5.5, 6.0, 6.5, 6.8 or 7.0 (optionally these pH values reached before, during or after, or before and during, administration), and optionally the buffer or a buffering agent or the pharmaceutically acceptable excipient comprises an inorganic salt, a citric acid, a sodium chloride, a potassium chloride, a sodium sulfate, a potassium nitrate, a sodium phosphate monobasic, a sodium phosphate dibasic or combinations thereof, and optionally the antacid comprises a calcium carbonate, a magnesium hydroxide, a magnesium oxide, a magnesium carbonate, an aluminum hydroxide, a sodium bicarbonate or a dihydroxyaluminum sodium carbonate.

In alternative embodiments, the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is capable of specifically binding to an animal (optionally a mammalian or a human cell), or is capable of specifically binding to a specific animal cell, and optionally the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is engineered to target a specific cell, tissue or organ, or diseased, infected or abnormal cell.

In alternative embodiments, an immune response is generated by display of epitopes or immunogens, or tolerogens, or immune response modulators, on the surface of the delivered or administered: (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), or by the inclusion of epitopes or immunogens, or tolerogens, or immune response modulators in the delivered or administered: (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v).

In alternative embodiments, the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is/are formulated per dose, or per serving, or per unit dosage at, or at a total daily dose of: between about 10(1)(or 10¹) and 10(20) plaque-forming units (PFUs), or between about 10(3) and 10(17) PFUs, or between about 10(5) and 10(12) PFUs, or between about 10(7) and 10(9) PFUs.

In alternative embodiments, the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), comprises, or contains within or upon, or carries, a payload or a composition,

wherein optionally the composition or the payload comprises: a drug; a modulator of transcytosis, endocytosis, exocytosis, receptor mediated endocytosis, non-specific binding, pinocytosis or macrocytosis in a eukaryotic cell; an immune response modulator, a epitope, an immunogen or a tolerogen; an antibiotic or a bacteriostatic agent; a cytotoxic agent; a nucleic acid (optionally an RNA (optionally an iRNA or miRNA), or an antisense nucleic acid, or a ribozyme, or a CRISPR or CRISPR/Cas9 nucleic acid, or a CRISPR/Cas9-gRNA complex for genome editing, or a DNA), wherein optionally the nucleic acid is derived from a phage, a bacterial or an animal, and optionally the nucleic acid is a synthetic or a recombinantly engineered nucleic acid, optionally the nucleic acid comprises a eukaryotic gene with the appropriate regulatory motifs, optionally promoters, such that the gene is expressed in a eukaryotic cell, optionally a gut cell; a genome or fragment thereof, wherein optionally the genome is derived from a phage or a bacterial genome; a carbohydrate, a protein or peptide, a lipid, an antibody or a small molecule; a label or tag or a fluorescent molecule or a radiopaque molecule; a magnetic particle; a radionucleotide; a carbohydrate binding domain (CBD) or a moiety or domain capable of binding to: a protein or peptide, a nucleic acid (optionally an RNA or a DNA), a lipid, a lipo-polysaccharide or a mucopolysaccharide; or, any combination thereof, wherein optionally the modulator of transcytosis, endocytosis, exocytosis, receptor mediated endocytosis, non-specific binding, pinocytosis or macrocytosis in the eukaryotic cell comprises or is an inhibitor or enhancer of transcytosis, endocytosis, exocytosis, receptor mediated endocytosis, non-specific binding, pinocytosis or macrocytosis, and optionally the inhibitor of transcytosis, endocytosis, exocytosis, receptor mediated endocytosis, non-specific binding, pinocytosis or macrocytosis is or comprises N-ethylmaleimide (NEM), chlorpromazine, filipin, colchicine, dynasore, Concanamycin C (Con C), eeyarestatin I, Golgicide A. Leptomycin B, levetiracetam, or brefeldin A (BFA), or an antibody that inhibits PIKFyve or a SNARE protein or an antibody that blocks SNARE assembly,

and optionally the nucleic acid is or comprises a small inhibitory RNA (siRNA), an antisense nucleic acid or RNA, or a CRISPR nucleic acid or CRISPR/Cas9 system comprising a synthetic guide RNA (gRNA) and/or a nuclease, or the nucleic acid encodes a protein or a small inhibitory RNA (siRNA), an antisense RNA, or a CRISPR nucleic acid or CRISPR/Cas9 system comprising a synthetic guide RNA (gRNA) and/or a nuclease,

and optionally the nucleic acid is contained in an expression vehicle or vector, and optionally the nucleic acid is operatively linked to a transcriptional control motif, which optionally can be a promoter and/or enhancer, optionally a tissue or cell specific, or constitutive, or inducible, promoter and/or enhancer,

and optionally the payload or composition is delivered to or released in, onto or into the eukaryotic cell, or is delivered or released into a eukaryotic cell subcellular compartment or an organelle,

and optionally the eukaryotic cell subcellular compartment or organelle is a cytoplasm, an endosome, an exosome, a liposome, a nucleus, a nucleosome, a golgi, an endoplasmic reticulum (ER) or a mitochondria,

and optionally the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is engineered to release the payload or composition into the eukaryotic cell, or eukaryotic cell subcellular compartment or organelle, or into a specific eukaryotic cell subcellular compartment or organelle,

and optionally the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is degraded in a lysosome, or is engineered or designed to be degraded in a lysosome.

In alternative embodiments, the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is or is derived from, or is substantially or partially derived from:

(a) a prokaryotic bacteriophage, optionally a bacterial or an Archaeal bacteriophage;

(b) a prokaryotic bacteriophage of the order Caudovirales or Ligamenvirales;

(c) a prokaryotic bacteriophage of the family Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttaviridae, Inoviridae, Leviviridae, Microviridae, Plasmaviridae or Tectivirus or a combination thereof;

(d) a Bacteroidetes-infecting phage or a class I filamentous phage, or an F1 or an Fd filamentous bacteriophage:

(e) a bacteriophage Qβ virus-like particle; or

(f) an Enterobacteria phage T4, a lambda phage, an M13 Inoviridae phage, a crAss phage, or a phage capable of infecting a mammalian or a human gut.

In alternative embodiments, the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is a chemically or structurally modified bacteriophage, phagemid or phage-like particle, and optionally the exterior (outer) surface of bacteriophage, phagemid or phage-like particle comprises:

(a) at least one heterologous:
(i) carbohydrate binding domain (CBD),
(ii) a moiety or domain capable of binding to a component of a mucus,
optionally a mucus of or derived from: a mammalian mucus membrane, a gut, a urinary, a reproductive, an animal or an environmental mucus,
optionally capable of binding to a mucus or mucus-like macromolecule, a mucin, a fatty acid, a phospholipid, a cholesterol, an elastin, a glycoprotein, a mucin glycoprotein or glycan, a mucin protein, a humic acid, a cellulose, a chitin, a high molecular weight (MW) polysaccharide, an N-acetylgalactosamine, an N-acetylglucosamine, a fucose, a galactose, a sialic acid (N-acetylneuraminic acid) a mannose, or any combination thereof,
and optionally the moiety or domain capable of binding to a component of a mucus directs or targets the bacteriophage, phagemid or phage-like particle to a specific region of a mucosal surface that overlaps with a bacterial host range, and optionally the specific region comprises a mucosal surface basal layer, a mucosal surface apical layer, a mucosal surface lumen, a mucus layer, or a mucosal surface having a concentration of between about 0% to 1% mucin, or between about 1% to 5%, or a mucin concentration of between about 1% to 10%,
and optionally the moiety or domain capable of binding to a component of a mucus directs or that targets the bacteriophage, phagemid or phage-like particle to a specific region of a mucosal surface allows the bacteriophage, phagemid or phage-like particle to reside or concentrate or persist in a specific region of the mucosal surface that overlaps with a bacterial host range,
and optionally the bacteriophage, phagemid or phage-like particle is adapted to a physico-chemical environment of the mucus or specific region of a mucosal surface, and the physico-chemical environment optionally comprises: a pH range of between about 6 to 8, a pH range of between about 4 to 10, a pH range of between about 1 to 12, an ionic concentration of between about 1 mg to 1000 mg, an ionic concentration of between about 1 μg to 1000 g, an ionic concentration of between about 1 pgm to 1000 kg, a temperature change of between about 35° C. to 42° C., a temperature change of between about 25° C. to 55° C., or a temperature change of between about 1° C. to 99° C.;
(iii) moiety or domain capable of binding to a protein or peptide, a protein or peptide (optionally an antibody or antigen binding fragment thereof, an antigen, an immunogen, a tolerogen), a glycoprotein, a nucleic acid (optionally an RNA or a DNA), a lipid or cholesterol, a lipopolysaccharide, a mucopolysaccharide, a gel, a hydrogel, a complex fluid, or a combination thereof, or
(iv) combination of any of (i) to (iii),

wherein optionally the heterologous CBD is a bacteriophage carbohydrate binding domain (CBD), and optionally the heterologous CBD is a CBD derived from a different species, genus, family or order of bacteriophage; or the CBD is a mammalian or a human CBD,

and optionally any of (i) to (iii) comprises or has structural homology to: a C-type lectin, a lectin, a bacteriodetes-associated carbohydrate-binding often N-terminal (BACON) domain, a Brefeldin A-inhibited guanine nucleotide-exchange factor for ADP-ribosylation factor (Big, optionally Big1, Big2, or Big3), a polycystic kidney disease domain (PKD), a Fibronectin type 3 homology domain (Fn3), a HYalin Repeat (HYR) domain, an Ig_2 domain, an immunoglobulin I-set domain, a carbohydrate-adherence domain, a mucus-binding protein, a glycan-binding protein, a protein-binding protein, a mucus-adhering protein or a mucus-adhering glycoprotein:

(b) additional homologous CBDs (more CBDs than found on a comparable wild type (WT) bacteriophage); or

(c) a combination of (a) and (b).

In alternative embodiments, the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), comprises or has contained therein a genome (optionally a substantially complete or a partial, or a genetically engineered or hybrid genome) that is altered such that after reproduction in a host cell (optionally a bacterial host cell), or in an in vitro system, the exterior (outer) surface of the bacteriophage comprises:

(a) at least one non-bacteriophage carbohydrate binding domain (CBD), and optionally the CBD is a mammalian or a human CBD;

(b) at least one heterologous bacteriophage CBD, wherein optionally the heterologous CBD is a CBD from a different species, genus, family or order of bacteriophage:

(c) more CBDs than found on a wild type (WT) (comparable) bacteriophage; or

(d) at least one moiety or domain capable of binding to a component of a mucus,
optionally a mucus of or derived from: a mammalian mucus membrane, a gut, a urinary, a reproductive, an animal or an environmental mucus,
optionally capable of binding to a mucus or mucus-like macromolecule, a mucin, a fatty acid, a phospholipid, a cholesterol, an elastin, a glycoprotein, a mucin glycoprotein or glycan, a mucin protein, a humic acid, a cellulose, a chitin, a high molecular weight (MW) polysaccharide, an N-acetylgalactosamine, an N-acetylglucosamine, a fucose, a galactose, a sialic acid (N-acetylneuraminic acid) a mannose, or any combination thereof,
and optionally the moiety or domain capable of binding to a component of a mucus directs or targets the phage to a specific region of a mucosal surface that overlaps with a bacterial host range, and optionally the specific region comprises a mucosal surface basal layer, a mucosal surface apical layer, a mucosal surface lumen, a mucus layer, or a mucosal surface having a concentration of between about 0% to 1% mucin, or between about 1% to 5%, or a mucin concentration of between about 1% to 10%,
and optionally the moiety or domain capable of binding to a component of a mucus directs or that targets the phage to a specific region of a mucosal surface allows the phage to reside or concentrate or persist in a specific region of the mucosal surface that overlaps with a bacterial host range,
and optionally the phage is adapted to a physico-chemical environment of the mucus or specific region of a mucosal surface, and the physico-chemical environment optionally comprises: a pH range of between about 6 to 8, a pH range of between about 4 to 10, a pH range of between about 1 to 12, an ionic concentration of between about 1 mg to 1000 mg, an ionic concentration of between about 1 μg to 1000 gram (g), an ionic concentration of between about 1 pgm (picogram) to 1000 kg, a temperature change of between about 35° C. to 42° C., a temperature change of between about 25° C. to 55° C., or a temperature change of between about 1° C. to 99° C.;

(e) at least one moiety or domain capable of binding to a protein or peptide, a protein or peptide (optionally an antibody or antigen binding fragment thereof, an antigen, an immunogen, a tolerogen), a glycoprotein, a nucleic acid (optionally an RNA or a DNA), a lipid or cholesterol, a lipopolysaccharide, a mucopolysaccharide, a gel, a hydrogel, a complex fluid, or a combination thereof; or

(f) any combination of (a) to (e),

and optionally any of (a) to (e) comprises or has structural homology to: a C-type lectin, a lectin, a bacteriodetes-associated carbohydrate-binding often N-terminal (BACON) domain, a Brefeldin A-inhibited guanine nucleotide-exchange factor for ADP-ribosylation factor (Big, optionally Big1, Big2, or Big3), a polycystic kidney disease domain (PKD), a Fibronectin type 3 homology domain (Fn3), a HYalin Repeat (HYR) domain, an Ig_2 domain, an immunoglobulin I-set domain, a carbohydrate-adherence domain, a mucus-binding protein, a glycan-binding protein, a protein-binding protein, a mucus-adhering protein or a mucus-adhering glycoprotein.

In alternative embodiments:

(a) the CBD is entirely, or substantially, a synthetic or non-natural CBD, optionally an antibody or antigen binding domain that specifically binds to a carbohydrate:

(b) the CBD is or comprises a protein having a carbohydrate-binding-like fold, which optionally comprises a seven-stranded beta-sandwich, or optionally is or comprises an immunoglobulin-like binding domain, or a protein domain comprising a 2-layer sandwich of between 7 and 9 antiparallel Î²-strands arranged in two Î²-sheets; (c) the CBD is or is derived from or has substantial structural identity (homology) to a mammalian or a human CBD;

(d) the bacteriophage is known or demonstrated to be toxic or lysogenic to a bacteria, or the bacteriophage is bactericidal or bacteriostatic, or the bacteriophage can treat, inhibit or prevent an infection, and optionally the bacteriophage is engineered to specifically bind to or target the bacteria,

wherein optionally the bacteriophages are bactericidal or bacteriostatic to a gram negative bacteria or a gram positive bacteria, and optionally the bacteriophage is engineered to specifically bind to or target the gram negative bacteria or gram positive bacteria,

and optionally the bacteria or infection is or is caused by an MSRA infection, a Staphylococcus, a Staphylococcus aureus, a Clostridium, a Clostridium difficile, a Escherichia coli, a Shigella, a Salmonella, a Campylobacter, a Chloerae, a Bacillus, a Yersinia or a combination thereof, and optionally the bacteriophage is engineered to specifically bind to or target the bacteria or

(e) the bacteriophage is made or identified by a process comprising: screening a plurality of bacteriophages for bactericidal or bacteriostatic properties against a bacterium of interest, and selecting the bacteriophages having a lysogenic or a bactericidal or bacteriostatic activity.

In alternative embodiments, the CBD is, or is derived from, or has substantial structural identity (homology to):

(a) a protein having a carbohydrate-binding-like fold, which optionally comprises a seven-stranded beta-sandwich, or optionally is or comprises an immunoglobulin-like binding domain, or comprises a protein domain comprising a 2-layer sandwich of between 7 and 9 antiparallel Î²-strands arranged in two Î²-sheets;

(b) a CBD, optionally an antibody or antigen binding fragment thereof, capable of specifically binding to a tumor associated carbohydrate antigen (TACA); or

(c) a carbohydrate-binding module family 1 (CBM1);

a carbohydrate-binding module family 2 (CBM2);

a carbohydrate-binding module family 3 (CBM3);

a carbohydrate-binding module family 4 (CBM4);

a carbohydrate-binding module family 5 (CBM5);

a carbohydrate-binding module family 6 (CBM6):

a carbohydrate-binding module family 7 (CBM7);

a carbohydrate-binding module family 8 (CBM8);

a carbohydrate-binding module family 9 (CBM9);

a carbohydrate-binding module family 10 (CBM10);

a carbohydrate-binding module family 11 (CBM11);

a carbohydrate-binding module family 12 (CBM12);

a carbohydrate-binding module family 13 (CBM13);

a carbohydrate-binding module family 14 (CBM14);

a carbohydrate-binding module family 15 (CBM15);

a carbohydrate-binding module family 16 (CBM16);

a carbohydrate-binding module family 17 (CBM17);

a carbohydrate-binding module family 18 (CBM18);

a carbohydrate-binding module family 19 (CBM19);

a carbohydrate-binding module family 20 (CBM20);

a carbohydrate-binding module family 21 (CBM21);

a carbohydrate-binding module family 25 (CBM25);

a carbohydrate-binding module family 27 (CBM27);

a carbohydrate-binding module family 28 (CBM28);

a carbohydrate-binding module family 33 (CBM33);

a carbohydrate-binding module family 48 (CBM48); or,

a carbohydrate-binding module family 49 (CBM49).

In alternative embodiments, provided are uses of: a composition, a product of manufacture, a food, a drink, a nutraceutical, a formulation, a pharmaceutical or a pharmaceutical preparation, wherein the composition, product of manufacture, food, drink, nutraceutical, formulation, pharmaceutical or pharmaceutical preparation is or comprises a composition, product of manufacture, food, drink, nutraceutical, formulation, pharmaceutical or pharmaceutical preparation as used in a method of any of the preceding claims, in the preparation or manufacture of a medicament for:

delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into an animal (optionally a mammal or a human),
optionally the delivering is into a tissue, the blood stream or lymphatic system of the animal, wherein optionally the delivering is ex vivo or in vivo, and optionally the animal is a mammal or a human,

delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into a eukaryotic cell,
wherein optionally the delivering comprises entering or injecting into a subcellular compartment or an organelle of the eukaryotic cell, and optionally the eukaryotic cell subcellular compartment or organelle comprises or is a cytoplasm, an endosome, an exosome, a liposome, a nucleus, a nucleosome, a golgi, an endoplasmic reticulum (ER) or a mitochondria, and optionally the eukaryotic cell is in or derived from a mammal or a human,

treating, ameliorating and/or preventing a bacterial or viral infection in an animal in vivo, wherein optionally the bacterial or viral infection in the animal is inside or outside of the gut of the animal,
wherein optionally the bacterial or viral infection comprises a gut, muscle, lung, liver, kidney or blood (sepsis) infection, or a secondary infection inside or outside of the gut,
and optionally the animal is a mammal or a human,

generating or modulating an immune response in an animal by delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into the animal (optionally a mammal or a human),
optionally delivering into a tissue, the blood stream or lymphatic system of the animal, or into a cell of the animal,
wherein optionally the immune response is a humoral (antibody) response, a cell-mediated immune response, or a tolerogenic immune (suppressing) response, and optionally the modulating of the immune response decreases, ameliorates or inhibits inflammation or an autoimmune reaction in the animal,
and optionally the decreasing, ameliorating or inhibiting of inflammation or the autoimmune reaction in the animal treats, ameliorates, decreases the severity of or inhibits a disease or condition caused by an inflammation or an autoimmune reaction or a disease or condition causing an inflammation or autoimmune reaction,
and optionally the immune response is modulated by inclusion of, release from or display on the surface of (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), an immunogen or a tolerogen,

treating, ameliorating and/or preventing a disease or condition in an individual in need thereof,
wherein optionally the disease or condition comprises obesity, diabetes, autism, a cystic fibrosis, an inflammation outside or outside of the gut,
and optionally the individual is an animal, a mammal, or a human, and/or

delivering a payload or a composition in vivo to an animal, or labelling, tagging or coating a cell in vivo in an animal,
wherein optionally the payload is delivered into a eukaryotic cell (intracellular delivery of the payload), wherein optionally the payload comprises a small molecule or a nucleic acid,

and optionally the animal is a mammal or a human.

In alternative embodiments, provided are therapeutic formulations of a composition, a food, a drink, a nutraceutical, a formulation, a pharmaceutical or a pharmaceutical preparation,

wherein the composition, food, drink, nutraceutical, formulation, pharmaceutical or pharmaceutical preparation is or comprises a composition, food, drink, nutraceutical, formulation, pharmaceutical or pharmaceutical preparation as used in a method of any of the preceding claims,

for use in:

delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into an animal (optionally a mammal or a human),
optionally the delivering is into a tissue, the blood stream or lymphatic system of the animal, wherein optionally the delivering is ex vivo or in vivo, and optionally the animal is a mammal or a human,

delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into a eukaryotic cell,
wherein optionally the delivering comprises entering or injecting into a subcellular compartment or an organelle of the eukaryotic cell, and optionally the eukaryotic cell subcellular compartment or organelle comprises or is a cytoplasm, an endosome, an exosome, a liposome, a nucleus, a nucleosome, a golgi, an endoplasmic reticulum (ER) or a mitochondrion, and optionally the eukaryotic cell is in or derived from a mammal or a human,

treating, ameliorating and/or preventing a bacterial or viral infection in an animal in vivo, wherein optionally the bacterial or viral infection in the animal is inside or outside of the gut of the animal,
wherein optionally the bacterial or viral infection comprises a gut, muscle, lung, liver, kidney or blood (sepsis) infection, or a secondary infection inside or outside of the gut,
and optionally the animal is a mammal or a human,

generating or modulating an immune response in an animal by delivering: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), into the animal (optionally a mammal or a human),
optionally delivering into a tissue, the blood stream or lymphatic system of the animal, or into a cell of the animal,
wherein optionally the immune response is a humoral (antibody) response, a cell-mediated immune response, or a tolerogenic immune (suppressing) response, and optionally the modulating of the immune response decreases, ameliorates or inhibits inflammation or an autoimmune reaction in the animal,
and optionally the decreasing, ameliorating or inhibiting of inflammation or the autoimmune reaction in the animal treats, ameliorates, decreases the severity of or inhibits a disease or condition caused by an inflammation or an autoimmune reaction or a disease or condition causing an inflammation or autoimmune reaction,
and optionally the immune response is modulated by inclusion of, release from or display on the surface of (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), an immunogen or a tolerogen,

treating, ameliorating and/or preventing a disease or condition in an individual in need thereof,
wherein optionally the disease or condition comprises obesity, diabetes, autism, a cystic fibrosis, an inflammation outside or outside of the gut,
and optionally the individual is an animal, a mammal, or a human, and/or

delivering a payload or a composition in vivo to an animal, or labelling, tagging or coating a cell in vivo in an animal,
wherein optionally the payload is delivered into a eukaryotic cell (intracellular delivery of the payload), wherein optionally the payload comprises a small molecule or a nucleic acid,
and optionally the animal is a mammal or a human.

The details of one or more embodiments as provided herein are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments as provided herein and are not meant to limit the scope of the invention as encompassed by the claims.

FIG. 1A-C illustrates that the transcytosis of bacteriophages occurs in a preferential apical-to-basal direction across diverse cell layers:

FIG. 1A schematically illustrates the experimental system to investigate phage transcytosis:

FIG. 1B graphically illustrates data showing the transcytosis of T4 phages across Madin-Darby canine kidney cells (MDCK) in either an apical-basal or basal-apical direction:

FIG. 1C graphically illustrates data showing the transcytosis of T4 phages across T84 cells (colon epithelial), CaCo2 cells (colon epithelial), A549 cells (lung epithelial), Huh7 (hepatocyte epithelial-like) and hBMec cells (brain endothelial);

as described in detail in Example 1, below.

FIG. 2A-D graphically illustrate data showing the rate and diversity of apical-to-basal phage transcytosis:

FIG. 2A graphically illustrates data showing the rate of T4 phage transcytosis across MDCK cells over a two-hour period;

FIG. 2B graphically illustrates data showing the transcytosis of T4 phage applied to MDCK cells at sequentially decreasing log 10 concentrations;

FIG. 2C graphically illustrates data showing the transcytosis of unprocessed and cleaned T4 phages across MDCK cells;

FIG. 2D graphically illustrates data showing the transcytosis of diverse phage types across MDCK cells;

as described in detail in Example 1, below.

FIG. 3A-B graphically illustrate data showing the inhibition of phage transcytosis and subcellular localization:

FIG. 3A graphically illustrates data showing the percent transcytosis of T4 phages across MDCK cells pre-treated with chemical inhibitors compared to a solvent control;

FIG. 3B graphically illustrates data showing the fractionation of MDCK cells treated with T4 phages for 18 hrs;

as described in detail in Example 1, below.

FIG. 4A-C illustrate the subcellular fractionation of MDCK cells treated with T4 phages:

FIG. 4A schematically illustrates the fractionation of cell lysate showing the six cellular fractions collected;

FIG. 4B graphically illustrates data showing that after the 5 min treatment of MDCK cells no phages were present in any cellular fractions, but phages were detected in the cell washes before fractionation:

FIG. 4C graphically illustrates data showing that after 18 hour treatment of MDCK cells phages were present in all major cellular fractions:

as described in detail in Example 1, below.

FIG. 5 illustrates an image of the visualization of intracellular phages, as described in detail in Example 1, below.

FIG. 6 illustrates images of MDCK cells treated with SYBR-Gold labeled T4 phages grown on Ibidi μ-Slide CorrSight™ Live, where red arrows show cells containing distinct SYBR-labeled puncta; blue arrows show cells with diffuse SYBR-labeled cytoplasm; as described in detail in Example 1, below.

FIG. 7A-B graphically illustrate a post-assay confluency test with Evans blue dye;

FIG. 7A graphically illustrates data showing the absorbance of Evans blue standard curve;

FIG. 7B graphically illustrates data showing the post-assay Evans blue dye concentrations from applied and collected wells;

as described in detail in Example 1, below.

FIG. 8A-C illustrate the subcellular fractionation of MDCK cells treated with T4 phages:

FIG. 8A schematically illustrates the fractionation of cell lysate showing the six cellular fractions collected;

FIG. 8A graphically illustrates data showing that after 5 min treatment of MDCK cells no phages were present in any cellular fractions, but phages were detected in the cell washes before fractionation;

FIG. 8B graphically illustrates data showing that after 18 hour treatment of MDCK cells phages were present in all major cellular fractions:

as described in detail in Example 1, below.

FIG. 9 schematically illustrates images of MDCK cells treated with SYBR-Gold labeled T4 phages grown on Ibidi μ-Slide CorrSight™ Live; red arrows indicated cells containing distinct SYBR-labeled puncta, blue arrows indicate cells with diffuse SYBR-labeled cytoplasm; as described in detail in Example 1, below.

FIG. 10A-K illustrates the visualization of intracellular phages; as further described Example 1, below:

FIG. 10A-I illustrates representative correlative micrographs of an MDCK cell stained with Hoechst (blue) and CellMask (red) after application of T4 phages stained with SYBR-gold (green): FIG. 10A illustrates SYBR-gold positive target cell was selected during confocal microscopy, then FIG. 10B illustrates the cell processed and aligned for inspection of the same structures by Transmission Electron Microscopy (TEM); FIG. 10C-I illustrates spatially aligned electron micrographs showing (FIG. 10G and FIG. 10H) SYBR-positive endomembrane structures, adjacent to (FIG. 10F and FIG. 10I) SYBR-negative virus particles.

FIG. 10J-K illustrates representative electron micrographs of: FIG. 10J illustrates extracellular and FIG. 10K illustrates intracellular virus particles found in CLEM samples; data illustrates in FIG. 10A is maximum projection between the 37^{th} and 43^{rd} optical sections (3.0 μm to 4.2 μm above coverslip surface); data illustrates in FIG. 10B is a distortion corrected TEM montage from the 47^{th} resin section (3670 nm sample depth, 85 nm thick) acquired at 25 kx; arrows indicate virus-like particles within membrane bound vesicles;

Scale bars: FIG. 10A-B, 10 μm; FIG. 10C-E, 500 nm; FIG. 10F-K, 100 nm.

FIG. 11A-J illustrate images where MDCK cells were treated with T4 phage labeled with both the DNA-complexing SYBR-gold stain (green) (as illustrated in FIG. 11C and FIG. 11H) and capsid conjugated Cy3 stain (red)(as illustrated in FIG. 11B and FIG. 11G) for either 30 min (as illustrated in FIG. 11A) or two hours (as illustrated in FIG. 11F) and imaged under confocal microscopy; cells were stained with Hoescht (blue) (as illustrated in FIG. 11D and FIG. 11I) and CellMask (white) (as illustrated in FIG. 1E and FIG. 11J) after application of T4 phage; 30 min treatment sample showed correlation of DNA and capsid fluorescence signals, compared with two hour treatment where there was a disassociation of fluorescence; scale bar: 10 μm; as further described Example 1, below.

FIG. 12A-B graphically illustrate data from a study of the subcellular fractionation of phage treated MDCK and A549 cells: FIG. 12A upper image graph illustrates data from the fractionation of MDCK cells treated with T4 phages (lysate) for 18 hrs, where cells were washed, lysed using the Lysosome Enrichment Kit and total number of phage in cells (Total Cell Lysate), total cell lysates were fractionated, ten cellular fractions were collected and split for either phage quantification by bacterial plating or Western blot analysis (lower images of FIG. 12A-B) of Golgi apparatus and endoplasmic reticulum; FIG. 12B upper image graph illustrates data from the fractionation of A549 cells treated with T4 phages for 18 hrs; scatter plots show median; error bars represent 95% confidence interval; bar plot shows mean; error bars show standard deviation; as further described Example 1, below.

FIG. 13 graphically illustrates data from a study of the inhibition of phage transcytosis: percent transcytosis of T4 phages across MDCK cells pretreated with chemical inhibitors Brefeldin A (5 pgm/ml), Wortmannin (100 nM), Bafilomycin (0.5 μM), Chloroquine (100 μM), and W-7 (100 μM), for 18 hrs compared to a solvent control; bar plot shows mean; error bars show standard deviation; as further described Example 1, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, provided are compositions, products of manufacture and methods for treating, ameliorating and preventing various infections, disorders and conditions in animals, e.g., mammals such as humans, in vivo, including genetically-predisposed and chronic disorders, by administration to an individual in need thereof a composition, a product of manufacture, a food, a drink, a nutraceutical, a formulation, a pharmaceutical or a pharmaceutical preparation comprising: (i) a bacteriophage (“phage”), wherein optionally the phage is a temperate phage or a lysogenic phage (ii) a prophage (optionally a tailocin, or a defective prophage where head and tail are absent but the prophage is otherwise adsorption-competent), a phagemid or a phage-like particle (optionally a phagocin), (iii) a general transducing agent (GTA), or a small, tailed bacteriophage-like particle, (iv) a Metamorphosis Associated Contractile structure (MACs), (v) a phage-derived product (optionally an endolysin, a holin, a lysozyme, or a tail fiber protein), or (vi) any combination of (i) to (v), including chemically or structurally modified, genetically engineered and/or synthetic forms thereof; which optionally comprise a payload for having the desired effect, for example, the payload can be a drug, an effector nucleic acid, an immunogen, a label. In alternative embodiments, compositions, products of manufacture and methods as provided herein are effective for delivering payloads of any kind (e.g., drugs, small molecules, nucleic acids, immune response modulators, labels) to an animal cell, or an animal in vivo, to have a desired effect.

In alternative embodiments, compositions, products of manufacture and methods as provided herein are used to treat, prevent or ameliorate an infection in an animal, e.g., a mammal, in vivo inside or outside of the gastrointestinal tract (inside or outside of the gut). In alternative embodiment, compositions and methods as provided herein are used to specifically target and/or bind to an animal, e.g., mammalian, cell, optionally in vivo, that is associated with or completely or partially causative of an infection, disease or a condition.

In alternative embodiment, compositions, products of manufacture and methods as provided herein are designed to target a particular cell, tissue or organ, e.g., in vivo. In alternative embodiments, compositions, products of manufacture and methods as provided herein comprise use of (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), to be specific for, or which are engineered, designed or constructed to be (e.g., by recombinant technology) specific for, or are capable of specifically targeting, a specific cell, tissue or organ in vivo, or a particular microbe, e.g., an infectious agent or a pathogen, or any microbe or bacteria that is pathogenic, or is associated with or completely or partially causative of an infection, disease or a condition.

In alternative embodiments, provided are compositions or products of manufacture, e.g., a drug delivery agent, a liposome or a micelle, a hydrogel, a dendrimer, a particle or a microparticle, a powder, a nanostructure or a nanoparticle, capable of targeting a specific cell, tissue or organ in vivo, or a microbe or bacteria, where in alternative embodiments the specific targeting is effected by incorporation of a component of (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), which is designed or constructed to be (e.g., by recombinant technology) specific for, or responsible for or is capable of, specifically targeting, a specific cell, tissue or organ in vivo, or a specific microbe or bacteria, which can be a particular infectious agent or pathogen, a microbe or a bacteria that is pathogenic, or is associated with or completely or partially causative of an infection or a condition, for example, a bacteria.

Provided herein in Example 1, below, are data providing evidence demonstrating that (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), as provided herein can: effectively and in sufficient amounts enter an animal, e.g., a mammal, e.g., an individual in need thereof, to deliver sufficient amounts of a payload, and optionally target to a specific cell, tissue or organ; or, enter into a tissue, the blood stream or a lymphatic system of the animal, e.g., a mammal, or to deliver a payload in vivo, for e.g., treating, ameliorating and/or preventing an infection, disease or condition in an individual in need thereof, where the infection, disease or condition is outside of the gut or gastrointestinal (GI) tract. Data evidence also demonstrates that phages, phagemids and phage-like particles and the like as provided herein can effectively and in sufficient amounts enter an animal, e.g., a mammal, to generate an immune response in the animal. In alternative embodiments, the immune response is generated by display of epitopes, tolerogens, drugs, or immunogens on the surface of, or within, the delivered (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v).

In alternative embodiment, compositions, products of manufacture and methods as provided herein (e.g., comprising the (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), as provided herein, optionally carrying a payload) target probiotic bacterial strains such that they can be engineered to constitutively produce phages and the like in the gut (or other organ or space) for delivery to e.g., epithelial cells.

In alternative embodiment, compositions, products of manufacture and methods as provided herein (e.g., comprising the (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), as provided herein), are designed to and are capable of delivering a payload to a cell, e.g., a gut cell; for example, the payload can be a nucleic acid, e.g., a eukaryotic gene, optionally with appropriate promoters or enhancers and the like, for recombinant gene expression in a eukaryotic, e.g., a gut cell. For example, phages, prophages and the like as provided herein can deliver synthetic gene networks to correct metabolic deficiencies such as galactosemia. In alternative embodiments, phages, prophages and the like as provided herein deliver an iRNA or miRNA, or CRISPR cassettes, e.g., CRISPR or CRISPR/Cas9 nucleic acids, or a CRISPR/Cas9-gRNA complex, to target genes for modifying the physiology of a cell, e.g., to a cancer cell to treat the cancer, or add a gene to a cell, or replace a defective gene in a cell, or knockout a gene in a cell.

In alternative embodiments, compositions, products of manufacture and methods as provided herein (e.g., comprising the (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), as provided herein), comprise a payload and can deliver the payload to a desired cell (e.g., a cell in the gut), e.g., deliver small compounds of therapeutics (e.g., drugs, nucleic acids) to cells. For example, in alternative embodiments, immunostimulatory compounds (e.g., vaccines, immunogens), or transcriptional activator proteins are delivered to cells to e.g., direct eukaryotic ribosomes to preferentially transcribe of phage-delivered genes, or anti-toxin compounds to prevent food-poisoning, and the like.

In alternative embodiments, compositions, products of manufacture and methods as provided herein (e.g., comprising the (i) a bacteriophage (“phage”), (ii) a prophage, a phagemid or a phage-like particle, (iii) a general transducing agent (GTA), or a small, tailed bacteriophage-like particle, (iv) a Metamorphosis Associated Contractile structure (MACs), (v) a phage-derived product, or (vi) any combination of (i) to (v), as provided herein), is delivered to and can traffic inside a eukaryotic cell (e.g., a gut cell) to target intracellular pathogens, parasites or agents, e.g., viral, bacterial, protozoan or fungal pathogens, e.g., Nocardia, Brucella, Francisella, Mycobacterium (e.g., Mycobacterium leprae and Mycobacterium tuberculosis), Legionella, Bartonella henselae, Francisella tularensis, Listeria monocytogenes, Salmonella enterica, Rhodococcus equi, Yersinia, Neisseria meningitidis Histoplasma capsulatum, Cryptococcus neoformans, Chlamydia, Rickettsia, Coxiella, Apicomplexa, Trpanosomatida or a Pneumocystis and the like, and kill or inactivate the intracellular pathogens or agents.

### Preservatives, Cryoprotectants, Lyoprotectants

In alternative embodiments, for practicing methods, products of manufacture, and compositions as provided herein, provided are a particle, a nanoparticle, a liposome, a tablet, a pill, a capsule, a gel, a geltab, a liquid, a powder, a suspension, a syrup, an emulsion, a lotion, an ointment, an aerosol, a spray, a lozenge, an ophthalmic preparation, an aqueous or a sterile or an injectable solution, a patch (optionally a transdermal patch or a medicated adhesive patch), an implant, a dietary supplement, an ice cream, an ice, a yogurt, a cheese, an infant formula or infant dietary supplement, a pasteurized milk or milk product or milk-comprising product, or a liquid preparation embodiment or candies, lollies, drinks and the like, there can be added various preservatives, cryoprotectants and/or lyoprotectants, including e.g., various polysaccharides or sugars (such as sucrose, fructose, lactose, mannitol), glycerol, polyethylene glycol (PEG), trehalose, glycine, glucose, dextran and/or erythritol, comprising e.g., (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), as provided herein.

In alternative embodiments, cryoprotectants that can be used are ethylene glycol, 1,2-Propanediol, Methylcelliosolve, Dimethyl Formamide, or Dimethylsulphoxide Methanol. In alternative embodiments, the content of these cryoprotectants are between about 1% and about 50% but generally between about 5% and about 15% is adequate.

In alternative embodiments, a compound or composition for practicing methods, products of manufacture and compositions as provided herein is frozen and/or is freeze-dried, or spray dried, or lyophilized, using any method known in the art. For example, a method for freeze-drying bacteriophage can be used as described by Puapermpoonsiri et al., Int J. Pharm. 2010 Apr. 15; 389(1-2):168-75, who used sucrose or poly(ethylene glycol) 6000 to make bacteriophage-comprising freeze-dried cakes; or a method for making freeze-dried formulations of bacteriophage encapsulated in biodegradable microsphere, as described by Puapermpoonsiri et al., European J. Pharmaceutics and Biopharmaceutics, Vol. 72, Issue 1, 2009, Pgs 26-33; or methods for making stable bacteriophage compositions or matrices, as described e.g., by Murthy et al. WO2006047870 A1, or U.S. Pat. No. 8,309,077.

In alternative embodiments, there are different types of final products that can be manufactured. In alternative embodiments, a product or a formulation for practicing methods, products of manufacture and compositions as provided herein is a liquid. In alternative embodiments, a product or a formulation as provided herein is frozen and kept at e.g. minus 80 degrees for usage later given a cryoprotectant is added.

### Biofilm Disrupting Compounds

In alternative embodiments, biofilm disrupting compounds are added into a composition or formulation for practicing methods, products of manufacture and compositions as provided herein, provided are (e.g., a food, drink, nutraceutical, formulation, pharmaceutical or pharmaceutical preparation). In alternative embodiments, in practicing the methods as provided herein, biofilm disrupting compounds are administered before or during (co-administered), or co-formulated with (e.g., in a multi-laminated tablet or capsule), or separately formulated, as the administered composition or formulation as provided herein. In alternative embodiments, disrupting biofilms are used to separate from the colonic mucosa an adherent polysaccharide/DNA—containing layer, the so-called “biofilm”.

In alternative embodiments, other biofilm disrupting components or agents also can be used, e.g., enzymes such as a deoxyribonuclease (DNase), a N-acetylcysteine, an auranofin, alginate lyase, glycoside hydrolase dispersin B; Quorum-sensing inhibitors e.g., ribonucleic acid III inhibiting peptide, Salvadora persica extracts, Competence-stimulating peptide, Patulin and penicillic acid; peptides—cathelicidin-derived peptides, small lytic peptide, PTP-7 (a small lytic peptide, see e.g., Kharidia (2011) J. Microbiol. 49(4):663-8, Epub 2011 Sep. 2), Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones and/or macrolide antibiotics or any combination thereof.

In alternative embodiments, biofilm disrupting components or agents are administered before and during the administration of a composition of this invention, e.g., as an antibacterial, in whatever format or formulation this may take place, for example, as a capsule.

In alternative embodiments, biofilm disrupting agents are added either before treatment and/or during and/or after treatment with a composition for practicing methods and compositions as provided herein. In alternative embodiments, biofilm disrupting agents are used singly or in combination.

In alternative embodiments, biofilm disrupting agents include particular enzymes and degrading substances including in N-acetylcysteine, deoxyribonuclease (DNase). Others would include Alginate, lyase and Glycoside hydrolase dispersin, Ribonucleic-acid-III inhibiting peptide (RIP), Salvadora persica extracts, Competence-stimulating peptide (CSP) Patulin (PAT) and penicillic acid (PA)/EDTA, Cathelicidin-derived peptides, Small lytic peptide, PTP-7, Nitric oxide, Chlorhexidine, Povidone-iodine (PI), Nanoemulsions, Lytic bacteriophages, Lactoferrin/xylitol hydrogel, Synthetic iron chelators, Cranberry components, Curcumin, Acetyl-11-keto-boswellic acid (AKBA). Barley coffee (BC) components, silver nanoparticles, azithromycin, clarithromycin, gentamicin, streptomycin and also Disodium EDTA. Ozone insufflations of the colon can also be used to disrupt the biofilm.

### Unit Dosage Forms and Formulations, Foods, and Delivery Vehicles

In alternative embodiments, a composition for practicing methods and compositions as provided herein (e.g., a particle, a nanoparticle, a liposome, a tablet, a pill, a capsule, a gel, a geltab, a liquid, a powder, a suspension, a syrup, an emulsion, a lotion, an ointment, an aerosol, a spray, a lozenge, an ophthalmic preparation, an aqueous or a sterile or an injectable solution, a patch (optionally a transdermal patch or a medicated adhesive patch), an implant, a dietary supplement, an ice cream, an ice, a yogurt, a cheese, an infant formula or infant dietary supplement, a pasteurized milk or milk product or milk-comprising product) can be further processed by, e.g., spray-drying or equivalent, e.g., spray-drying in an inert gas or freeze-drying under similar conditions, thus ending up with a powdered product.

In alternative embodiments, a composition as provided herein can be formulated for enteral or parenteral administration, e.g., to reach the systemic circulation, or for local delivery (e.g., for administration to skin, ears, teeth), as a topical for e.g., infections, as an inhalant, e.g., for inhalation of phages for the treatment of e.g., lung infections, as described e.g., by Ryan et al. J Pharm Pharmacol. 2011 October; 63(10):1253-64.

In alternative embodiments, a composition is manufactured, labelled or formulated as a liquid, a suspension, a spray, a gel, a geltab, a semisolid, a tablet, or sachet, a capsule, a lozenge, a chewable or suckable unit dosage form, or any pharmaceutically acceptable formulation or preparation. In alternative embodiments, a composition as provided herein is incorporated into a food or a drink (e.g., a yogurt, ice cream, smoothie), a candy, sweet or lolly, or a feed, a nutritional or a food or feed supplement (e.g., liquid, semisolid or solid), and the like.

For example, bacteriophage used to practice the invention can be encapsulated as described, e.g., by Murthy et al. in US 2012-0258175 A1. A composition as provided herein can be manufactured, labelled or formulated as an orally disintegrating tablet as described e.g., in U.S. Pat. App. Publication No. 20100297031. A composition as provided herein can be a polyol/thickened oil suspension as described in U.S. Pat. No. (USPN) 6,979,674; 6,245,740. A composition as provided herein can be encapsulated, e.g., encapsulated in a glassy matrix as described e.g., in U.S. Pat. App. Publication No. 20100289164; and U.S. Pat. No. 7,799,341. A composition as provided herein can be manufactured, labelled or formulated as an excipient particle, e.g., comprising a cellulosic material such as microcrystalline cellulose in intimate association with silicon dioxide, a disintegrant and a polyol, sugar or a polyol/sugar blend as described e.g., in U.S. Pat. App. Publication No. 20100285164. A composition as provided herein can be manufactured, labelled or formulated as an orally disintegrating tablet as described e.g., in U.S. Pat. App. Publication No. 20100278930. A composition as provided herein can be manufactured, labelled or formulated as a spherical particle, as described e.g., in U.S. Pat. App. Publication No. 20100247665, e.g., comprising a crystalline cellulose and/or powdered cellulose. A composition as provided herein can be manufactured, labelled or formulated as a rapidly disintegrating solid preparation useful e.g. as an orally-disintegrating solid preparation, as described e.g., in U.S. Pat. App. Publication No. 20100233278. A composition as provided herein can be manufactured, labelled or formulated as a solid preparation for oral application comprising a gum tragacanth and a polyphosphoric acid or salt thereof, as described e.g., in U.S. Pat. App. Publication No. 20100226866.

A composition as provided herein can be manufactured, labelled or formulated using a water soluble polyhydroxy compound, hydroxy carboxylic acid and/or polyhydroxy carboxylic acid, as described e.g., in U.S. Pat. App. Publication No. 20100222311. A composition as provided herein can be manufactured, labelled or formulated as a lozenge, or a chewable and suckable tablet or other unit dosage form, as described e.g., in U.S. Pat. App. Publication No. 20100184785.

A composition as provided herein can be manufactured, labelled or formulated in the form of an agglomerate, as described e.g., in U.S. Pat. App. Publication No. 20100178349. A composition as provided herein can be manufactured, labelled or formulated in the form of a gel or paste, as described e.g., in U.S. Pat. App. Publication No. 20060275223. A composition as provided herein can be manufactured, labelled or formulated in the form of a soft capsule, as described e.g., in U.S. Pat. No. 7,846,475, or 7,763,276.

The polyols used in compositions as provided herein can be micronized polyols, e.g., micronized polyols, e.g., as described e.g., in U.S. Pat. App. Publication No. 20100255307, e.g., having a particle size distribution (d₅₀) of from 20 to 60 μm, and a flowability below or equal to 5 s/100 g, or below 5 s/100 g.

In practicing the invention, a wide variation of bacteriophage can be administered, for example, in some aspects, a smaller dosage can be administered because phage (i.e., bacteriophage) can replication in the host, i.e., in the individual to which a composition as provided herein is administered. In alternative embodiments, compositions as provided herein, including bacteriophages, phagemids or phage-like particles as provided herein, are formulated per dose, or per serving, or per unit dosage at, or at a total daily dose of between about 10(1) (or 10¹) and 10(20) plaque-forming units (PFUs), or between about 10(3) and 10(17) PFUs, or between about 10(5) and 10(12) PFUs, or between about 10(7) and 10(9) PFUs.

### Gradual or Delayed Release Formulations

In alternative embodiments, provided are methods using compositions formulated for delayed or gradual enteric release comprising at least one active agent (e.g., a composition, a formulation or a pharmaceutical preparation as provided herein) formulated with a delayed release composition or formulation, coating or encapsulation. In alternative embodiments, formulations or pharmaceutical preparations as provided herein and used in methods provided herein are designed or formulated for delivery of active ingredient (e.g., a bacteriophage) into the distal small bowel and/or the colon. Thus, for this embodiment, it is important to allow the active ingredient to pass the areas of danger, e.g., stomach acid and pancreatic enzymes and bile, and reach undamaged to be viable in the distal small bowel and especially the colon. In alternative embodiments, a formulation or pharmaceutical preparation as provided herein is a liquid formulation, a microbiota-comprising formulation as provided herein and/or a frozen or a freeze-dried version thereof. In alternative embodiments, preferably for the encapsulated format, all are in powdered form.

In alternative embodiments, compositions as provided herein are formulated for delayed or gradual enteric release using cellulose acetate (CA) and polyethylene glycol (PEG), e.g., as described by Defang et al. (2005) Drug Develop. & Indust. Pharm. 31:677-685, who used CA and PEG with sodium carbonate in a wet granulation production process.

In alternative embodiments, compositions as provided herein are formulated for delayed or gradual enteric release using a hydroxypropylmethylcellulose (HPMC), a microcrystalline cellulose (MCC) and magnesium stearate, as described e.g., in Huang et al. (2004) European J. of Pharm. & Biopharm. 58: 607-614).

In alternative embodiments, compositions as provided herein are formulated for delayed or gradual enteric release using e.g., a poly(meth)acrylate, e.g. a methacrylic acid copolymer B, a methyl methacrylate and/or a methacrylic acid ester, a polyvinylpyrrolidone (PVP) or a PVP-K90 and a EUDRAGIT® RL PO™, as described e.g., in Kuksal et al. (2006) AAPS Pharm. 7(1), article 1, E1 to E9.

In alternative embodiments, compositions as provided herein are formulated for delayed or gradual enteric release as described in U.S. Pat. App. Pub. 20100239667. In alternative embodiments, the composition comprises a solid inner layer sandwiched between two outer layers. The solid inner layer can comprise a formulation or pharmaceutical preparation as provided herein and one or more disintegrants and/or exploding agents, one of more effervescent agents or a mixture. Each outer layer can comprise a substantially water soluble and/or crystalline polymer or a mixture of substantially water soluble and/or crystalline polymers, e.g., a polyglycol. These can be adjusted in an exemplary composition as provided herein to achieve delivery of the living components of an FMT distally down the bowel.

In alternative embodiments, compositions as provided herein are formulated for delayed or gradual enteric release as described in U.S. Pat. App. Pub. 20120183612, which describes stable pharmaceutical formulations comprising active agents in a non-swellable diffusion matrix. In alternative embodiments, a formulation or pharmaceutical preparation as provided herein is released from a matrix in a sustained, invariant and, if several active agents are present, independent manner and the matrix is determined with respect to its substantial release characteristics by ethylcellulose and at least one fatty alcohol to deliver bacteria distally.

In alternative embodiments, a formulation or pharmaceutical preparation as provided herein is formulated for delayed or gradual enteric release as described in U.S. Pat. No. 6,284,274, which describes a bilayer tablet containing an active agent (e.g., an opiate analgesic), a polyalkylene oxide, a polyvinylpyrrolidone and a lubricant in the first layer and a second osmotic push layer containing polyethylene oxide or carboxy-methylcellulose.

In alternative embodiments, a formulation or pharmaceutical preparation as provided herein is formulated for delayed or gradual enteric release as described in U.S. Pat. App. Pub. No. 20030092724, which describes sustained release dosage forms in which a nonopioid analgesic and opioid analgesic are combined in a sustained release layer and in an immediate release layer, sustained release formulations comprising microcrystalline cellulose, EUDRAGIT RSPO™, CAB-O-SIL™, sodium lauryl sulfate, povidone and magnesium stearate.

In alternative embodiments, a formulation or pharmaceutical preparation as provided herein is formulated for delayed or gradual enteric release as described in U.S. Pat. App. Pub. 20080299197, describing a multi-layered tablet for a triple combination release of active agents to an environment of use, e.g., in the GI tract. In alternative embodiments, a multi-layered tablet is used, and it can comprise two external drug-containing layers in stacked arrangement with respect to and on opposite sides of an oral dosage form that provides a triple combination release of at least one active agent. In one embodiment, the dosage form is an osmotic device, or a gastro-resistant coated core, or a matrix tablet, or a hard capsule. In these alternative embodiments, the external layers may contain biofilm dissolving agents and internal layers the living bacteria.

In alternative embodiments, a formulation or pharmaceutical preparation as provided herein is formulated as multiple layer tablet forms, e.g., where a first layer provides an immediate release of a formulation or pharmaceutical preparation as provided herein and a second layer provides a controlled-release of another (or the same) formulation or pharmaceutical preparation as provided herein, or another active agent, as described e.g., in U.S. Pat. No. 6,514,531 (disclosing a coated trilayer immediate/prolonged release tablet), U.S. Pat. No. 6,087,386 (disclosing a trilayer tablet), U.S. Pat. No. 5,213,807 (disclosing an oral trilayer tablet with a core comprising an active agent and an intermediate coating comprising a substantially impervious/impermeable material to the passage of the first active agent), and U.S. Pat. No. 6,926,907 (disclosing a trilayer tablet that separates a first active agent contained in a film coat from a core comprising a controlled-release second active agent formulated using excipients which control the drug release, the film coat can be an enteric coating configured to delay the release of the active agent until the dosage form reaches an environment where the pH is above four).

In alternative embodiments, a formulation or pharmaceutical preparation as provided herein is formulated for delayed or gradual enteric release as described in U.S. Pat. App. Pub. 20120064133, which describes a release-retarding matrix material such as: an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinylacetates, polyvinylacetate copolymers or any combination. In alternative embodiments, spherical pellets are prepared using an extrusion spheronization technique, of which many are well known in the pharmaceutical art. The pellets can comprise one or more formulations or pharmaceutical preparations as provided herein, e.g., the liquid preparation embodiment.

In alternative embodiments, a formulation or pharmaceutical preparation as provided herein is formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candellia wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

In alternative embodiments, a formulation or pharmaceutical preparation as provided herein is a powder that can be included into a tablet or a suppository. In alternative embodiments, a formulation or pharmaceutical preparation as provided herein can be a ‘powder for reconstitution’ as a liquid to be drunk or otherwise administered. In alternative embodiments, a formulation or pharmaceutical preparation as provided herein is micro-encapsulated, formed into tablets and/or placed into capsules, especially enteric-coated capsules.

### Buffers and Antacids

In alternative embodiments, in practicing the methods as provided herein, buffers or antacids are administered before or during (co-administered), or co-formulated with a composition or formulation as provided herein. For example, in alternative embodiments, a composition or formulation as provided herein and a buffer or antacid are co-formulated, e.g., as multiple layer tablet form or as a multi-laminated tablet or capsule. In alternative embodiments of methods as provided herein, buffers or antacids are separately formulated. In alternative embodiments, the antacid, buffer or buffering agent is administered (optionally before, during or after, or before and during, administration) to raise the pH of the stomach in the individual to between about 2.5 and 7, or between about 3 and 6.5, or to about 5.0, 5.5, 6.0, 6.5, 6.8 or 7.0 (optionally these pH values reached before, during or after, or before and during, administration). In alternative embodiments, the buffer or a buffering agent or the pharmaceutically acceptable excipient comprises an inorganic salt, a citric acid, a sodium chloride, a potassium chloride, a sodium sulfate, a potassium nitrate, a sodium phosphate monobasic, a sodium phosphate dibasic or combinations thereof. In alternative embodiments, the antacid comprises a calcium carbonate, a magnesium hydroxide, a magnesium oxide, a magnesium carbonate, an aluminum hydroxide, a sodium bicarbonate or a dihydroxyaluminum sodium carbonate.

### Feeds, Drinks, Candies, Nutritional or a Food or Feed Supplements

In alternative embodiments, a formulation or pharmaceutical preparation as provided herein, or used in a method provided herein, is incorporated into a food, a feed, a candy (e.g., a lollypop or a lozenge) a drink, a nutritional or a food or feed supplement (e.g., liquid, semisolid or solid), and the like, as described e.g., in U.S. Pat. App. Publication No. 20100178413. In one embodiment, a formulation or pharmaceutical preparation as provided herein is incorporated into (manufactured as) a beverage as described e.g., in U.S. Pat. No. 7,815,956. For example, a composition as provided herein is incorporated into a yogurt, an ice cream, a milk or milkshake, a “frosty”, “snow-cone”, or other ice-based mix, and the like.

In alternative embodiments, a formulation or pharmaceutical preparation as provided herein is a freeze-dried powder form added to a food, e.g., a yogurt, an ice cream, a milk or milkshake, a “frosty”, “snow-cone”, or other ice-based mix, and the like. In one form of this invention it can be kept in a lid-storage (e.g., of a yogurt or ice cream) such that when it is twisted the powder falls into the product or formulation (e.g., yoghurt or ice cream) and then it can be stirred so as not to have the powder ferment ‘standing on the shelf’. Various flavourings can be added. In alternative embodiments, this is particularly important for administration of a composition as provided herein, e.g., a wild type microbiota or a cultured bacteria, to a very young individual and/or a patient with autism or related disease or condition.

In alternative embodiments, these exemplary products are important when administered to children or babies who may have acquired various pathogenic or abnormal bacteria, e.g., E. coli, Clostridia or Disulfovibrio, e.g., as in autism.

### Packaging

Compositions as provided herein and used to practice methods as provided herein (e.g., a product of manufacture, food, drink, nutraceutical, formulation, pharmaceutical or pharmaceutical preparation), including preparations, formulations and/or kits, comprise combinations of ingredients, as described herein. In alternative embodiments, these combinations can be mixed and administered together, or alternatively, they can be an individual member of a packaged combination of ingredients, e.g., as manufactured in a separate package, kit or container; or, where all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs as provided herein) combination of active ingredients) as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack as provided herein comprises a moulded PVC base, with raised areas (the “blisters”) to contain the tablets, pills, etc. comprising the combinations as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided are methods of packaging where the compositions comprising combinations of ingredients as provided herein are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. as provided herein are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components (e.g., is a multi-ingredient combination as provided herein): a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

### Blister Packaging

In alternative embodiments, combinations of ingredients of compositions as provided herein or used to practice methods provided herein, or combinations of ingredients for practicing methods as provided herein, can be packaged alone or in combinations, e.g., as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of drugs designed to dissolve immediately in the mouth of a patient. This exemplary process comprises having the drug combinations as provided herein prepared as an aqueous solution(s) which are dispensed (e.g., by measured dose) into an aluminium (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system gives tablets an identification mark by embossing a design into the alufoil pocket that is taken up by the tablets when they change from aqueous to solid state. In one aspect, individual ‘push-through’ blister packs/packettes are used, e.g., using hard temper aluminium (e.g., alufoil) lidding material. In one aspect, hermetically-sealed high barrier aluminium (e.g., alufoil) laminates are used. In one aspect, any as provided herein's products of manufacture, including kits or blister packs, use foil laminations and strip packs, stick packs, sachets and pouches, peelable and non-peelable laminations combining foil, paper, and film for high barrier packaging.

In alternative embodiments, any as provided herein's multi-ingredient combinations or products of manufacture, including kits or blister packs, include memory aids to help remind patients when and how to take the drug. This safeguards the drug's efficacy by protecting each tablet, geltab or pill until it's taken; gives the product or kit portability, makes it easy to take a dose anytime or anywhere.

The invention will be further described with reference to the following examples; however, it is to be understood that the invention is not limited to such examples.

### EXAMPLES

### Example 1: Exemplary Phage Compositions and Methods of Using them

This example provides data evidence demonstrating that a (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), as provided herein can effectively and in sufficient amounts enter an animal, e.g., a mammal, e.g., an individual in need thereof, to deliver sufficient amounts of, and optionally target to a specific cell, tissue or organ, the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), into the blood stream or lymphatic system of the animal, e.g., a mammal, or deliver the (i) bacteriophage (“phage”) (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), to a tissue or organ of the animal, e.g., a mammal, in vivo, for e.g., treating, ameliorating and/or preventing an infection, disease or condition in an individual in need thereof, where the infection, disease or condition is outside of the gut or gastrointestinal (GI) tract.

Here we show that bacteriophage transcytosis across diverse epithelial cell layers was both irregular but repeatable, with a strong apical-to-basolateral membrane directionality. Bacteriophages transit through the Golgi and accessed microsomal fractions of the cell, suggesting free uptake by endocytosis as the mechanism to access the body. Using experimental data, we estimate that thirty-billion bacteriophage particles are transcytosed by the average human body every day, with comparable ingress via a ‘leaky gut’ requiring significant intestinal damage. The transcytosis of bacteriophage into the body is a natural and ubiquitous process that may have important implications, including vertical transmission of gut phages and immunostimulatory effects on the body.

### Methods

### Bacterial Strains, Phage Stocks, Tissue Culture Cell Lines and Growth Conditions

Escherichia coli B strain HER 1024 was grown in LB (10 g tryptone, 5 g yeast extract, 10 g NaCl, in 1 L dH₂O) at 37° C. shaking overnight and used to propagate and quantify bacteriophages T4, T3, T5 and T7. Bacillus subtilis 168WT was grown in TY broth (10 g tryptone, 5 g yeast extract, 5 g NaCl, 10 mM MgSO₄, 100 μM MnSO₄, in 1 L dH₂O) at 37° C. shaking for 6-8 hrs and used to propagate and quantify bacteriophages SP01 and SPP1. Salmonella typhimurium LT2 was grown in LB at 37° C. shaking overnight and used to propagate and quantify bacteriophages P22. All phage lysates were purified and cleaned of bacterial endotoxins according to the Phage-On-Tap protocol (1).

All tissue culture cells lines were grown at 37° C. and 5% CO₂ and supplemented with 1% Penicillin/Streptomycin (Mediatech, Inc., Tewksbury, Mass.). MDCK.2 cells were grown in Eagles's Minimal Essential Media with 10% Fetal Bovine Serum (FBS), T84 cells were grown in Ham's F12 medium and Dulbecco's modified Eagle's medium with 2.5 mM L-glutamine with 10% FBS, CaCo2 cells were grown in Eagles's Minimal Essential Media with 10% FBS, A549 and Huh7 cells were grown in F-12K medium with 10% FBS, hBMec cells were grown in RPMI medi with 10% nuSerum (Corning, N.Y.) and 1% NEAA (GIBCO, Walktham, Mass.).

### Transwell Experimental Setup

TRANSWELL® PET 12 well plates with 0.4 μm pore size (Corning, N.Y.) were used for all transcytosis assays. All cells were seeded at a density of 0.5-1×10⁶ cells per well and allowed to grow to confluency (3-5 days). For apical-to-basal transcytosis the apical wells were incubated in Hanks Buffered Salt Solution (HBSS) at pH 6.0 and basal cells in HBSS at pH 7.4 for two hours to mimic pH-dependent uptake (2). For basal-to-apical transcytosis the buffers were switched. Bacteriophages were applied with the HBBS pH 6.0 buffer, incubated with cells for two hours and phage from both apical and basal cell layers quantified by plating with bacterial host.

Cell layer confluency of all transwell experiments were measured in three separate ways to ensure phage transcytosis across the cell layer, rather than by paracellular transport. Firstly, a visual inspection using a phase-contrast microscope. Secondly, transepithelial resistances (TER) of all cell lines were measured (World Precision Instruments, Sarasota Fla.), with the acceptable range of measurements between 150-200 Ω*cm². TER measurements were taken before and after all transcytosis experiments to ensure cell confluency and polarization had been reached and maintained. Finally, 250 μL of HBSS buffer with 25 μL of Evans blue dye was added to the apical chambers of all transwells post-assay and incubated with cells at 37° C. for 2 hrs. Basal chambers were collected and absorbance was measured (620 nm) using a spectrophotometer and was compared against an Evans blue dilution curve (Fig S1). The presence of dye in the basal chamber was indicative of a non-confluent cell layer and data from these wells were discarded.

### Subcellular Fractionation

MDCK cells grown to confluence were incubated with T4 phages for either 5 minutes or 18 hours. Cell layers were then extensively washed with DPBS and subjected to microsomal fractionation (3), using the Lysosomal Enrichment™ kit for Tissue and Cultured Cells according to manufacturer's instructions (Thermo-Fisher). Briefly, approximately 200 mg of cells were harvested with trypsin and centrifuged for 2 min at 850×g. Lysosome enrichment reagent A containing a protease inhibitor cocktail (CalBioChem) was added to pelleted cells and subjected to a 2 min incubation on ice. After incubation, cells were then sonicated 15 times to lyse the cells, followed by addition of Lysosome enrichment reagent B containing a protease inhibitor. Cells were then centrifuged for 10 min at 500×g at 4° C. The supernatant was then collected and the final concentration was altered to 15% with OPTIPREP CELL SEPARATION MEDIA™. Samples were then loaded on a discontinuous OPTIPREP™ gradient from 60%. 30%, 27%, 23%. 20% to 17% om a 13.2 mL ultracentrifugation tube (Beckman-coulter) and centrifuged in a SW 41 Ti rotor at 145,000×g for 2 hours at 4° C. After ultracentrifugation, the lysosomal fraction was isolated from the top of the gradient, and all other microsomal fractions were isolated (Fig S2). All microsomal fractions were washed using two volumes of DPBS in a microcentrifuge tube at 17,000×g for 30 min at 4° C. Microsomal pellets were then washed with DPBS and centrifuged again at 17,000×g for 30 min at 4° C. Pellets were lysed with 0.1 volumes chloroform for 10 min, followed by centrifugation at 17,000×g for 5 min. Supernatants were then plated with bacterial hosts and phages quantified.

### Graphing and Statistics

Graphing and statistical analyses were performed using GRAPHPAD PRISM 7™ (GraphPad Prism; GraphPad Software). Individual data points, medians and standard deviations were reported where possible (4). Both non-parametric and parametric statistical analyses were performed, although most data did not pass a normality test.

### Phage Transcytosis Model

Phage transcytosis rate. We calculate the transcytosis rate per unit time, surface area (wall), and concentration of T4 phages and T84 gut epithelial cells. In a first approximation, this leads to:

$${r}_{tr} = \frac{{φ}_{lo} {V}_{lo}}{{S}_{lo}} \frac{1}{{φ}_{up}} \frac{1}{{t}_{in}} \approx 0.166 μ m \text{/} h \qquad Eq . ( S1 )$$

where ϕₗₒ is the basal concentration of phages, Vₗₒ is the volume of the lower compartment, Sₗₒ is the surface area of a single Transwell, ϕᵤₚ is the apical concentration of phages, and tᵢₙ is the time of incubation. The first term (ϕₗₒVₗₒ,Sₗₒ=σ) represents the number of phages that accomplished transcytosis per unit area of the Transwell. This number depends on how many phages contact the epithelial cells on the apical side. Thus, we divide by the apical concentration of phages (ϕᵤₚ). This assumes that the transcytosis mechanism is independent on the number of phages contacting the cell and being transported at a given time. The duration of the experiment will also impact the number of phages that are counted in the basal part of the cell. Thus, we divide by the time of incubation (tᵢₙ), which assumes that the rate of the transcytosis mechanism is approximately constant during the time scale of the experiment. This leads to a transcytosis rate, rₜᵣ, of 0.166 μm/h.

Number of transcytosed phages in humans. We estimate the number of phages that are being transcytosed in one day (24 hours) in the average human body. To calculate this number we combine the experimentally derived transcytosis rate with physiological data. Using the model in Eq. (S1), the number of phages transcytosed in humans per day is:

$${Φ}_{tr}^{day} = {r}_{tr} {S}_{li} \frac{{Φ}_{li}}{{V}_{li}} 24 h \approx 7.04 \cdot {10}^{9} phages \text{/} day \qquad Eq . ( S2 )$$

In this equation, we multiply the transcytosis rate (rₜᵣ) by the surface area of the large intestine (Sₗᵢ) times the concentration of phages in the intestine (Φₗᵢ/Vₗᵢ) times 24 hours (1 day). In this way, we estimate that there are seven billion phages that penetrate the human body per day using the transcytosis route.

Mucus factor. We then assume a 4.4-fold increase of phage numbers associated with mucosal surfaces in the large intestine, giving a total of

Φ_{tr,m}^{day}≈4.4×Φₜᵣ^{day}≈30.97·10⁹ phages/day Eq. (S3)

Thus, there are approximately thirty-one billion phages transcytosed by the human body per day. The constants used in this model are reported in Table S5 (physiological parameters in the large intestine), Table S6 (parameters of the transcytosis experiment), and Table S7 (model results).

Transcytosis in MCDK cells. The results above were based on the experiments done in T84 cells. Here we calculate the factor required to extend the results to Madin-Darby canine kidney cells (MCDK). In T84 cells, when 4.7×10⁷ phage ml⁻¹ are applied to the apical side and 7.9×10³ phage ml⁻¹ were recovered in the basal side. This leads to a raw transcytosis ratio of 1.48×10¹. In MCDK cells, 3.2×10⁷ phage ml⁻¹ were applied to the apical side, and 1.9×10⁴ phage ml⁻¹ were recovered in the basal side, that is, a raw transcytosis ratio of 2.9×10⁻⁴. Thus, the transcytosis ratio is 1.96 times higher in MCDK cells than in T84 cells. To estimate the transcytosis in MCDK we multiply our modelled results of T84 by ƒ≈1.96, giving a transcytosis rate, rₜᵣ, of 0.325 μm/h.

### Phage Leaky-Gut Model

We assume phages can bypass confluent epithelial layers at sites of inflammation caused by cellular damage and punctured vasculature. Here we introduce a mathematical model to estimate the flux of phages penetrating the body using this route. The constants used in and the values obtained from the model are summarized in Table S8.

Leaky-gut model upper bound limit. In a first approximation, we consider that every damaged region in the gut is equivalent to removing an entire epithelial cell, thus opening a channel 40 um long (Table S5), and we assume that the channel is filled with the same fluid as the gut surface. This allows phages to diffuse from the gut to the lymphatic and blood circulatory system. To obtain an upper bound limit to the number of phages penetrating the body by this mechanism, we neglect entropic effects associated to the section or number of channels, that is, we consider that multiple punctured points or a single hole with the same effective section lead to the same leaking. Under this assumption, phages will have the same diffusion constant both in the gut and in the channel. In the upper bound limit, we consider that phages diffuse as if they were in water at the body temperature (37° C.). The diffusion constant, D_{w}, as stated in the Einstein-Smoluchowski equation, is the ratio of the thermal energy, k_{B}T, and the friction coefficient of phages in water, γ_{w}, as given by Eq. (S4).

$${D}_{w} = \frac{{k}_{B} T}{{γ}_{w}} = \frac{{k}_{B} T}{6 π {R}_{φ} {v}_{w}} \approx 10.95 μ {m}^{2} \text{/} s \qquad Eq . ( S4 )$$

Here k_{B} is the Boltzmann constant. The friction constant is obtained by applying the Stokes-Einstein relation as shown in the denominator of the third term in Eq. (S4), where R_{ϕ} is the effective radius of the phage; most phages are quasi-spherical and have a similar size to lambda phage, so we assume an effective radius of R_{ϕ}≈30 nm. The viscosity of water at body temperature is v_{w}=0.6913 mPa s I (8). This leads to an approximate phage diffusion constant of 11 μm²/s, that is, phages cover an effective region of radius √11≈3.3 μm per second.

The flux of phages penetrating the body, J_{w}, is proportional to the diffusion coefficient, D_{w}, and the gradient of phage concentration in the transport channel as given by the Fick's law:

$${J}_{w} = - {D}_{w} \frac{\partial φ}{\partial x} \qquad Eq . ( S5 )$$

To calculate the flux, we need to determine the concentration profile of phages across the channel. This profile is determined by the diffusion equation:

$$\frac{\partial φ}{\partial t} = {D}_{w} \frac{{\partial }^{2} φ}{\partial {x}^{2}} \qquad Eq . ( S6 )$$

We consider that the side of the channel in the gut provides a constant supply of phages, ϕ(0)=ϕ₀, while in the other side of the channel (blood stream or lymphatic system) the phages do not accumulate, ϕ(H)=0, where H is the “length” of the channel (height in Table S5). This will eventually lead to a stable concentration profile that does not change in time, i.e., it is stationary:

$$\frac{\partial φ}{\partial t} = 0 \to \frac{{\partial }^{2} φ}{\partial {x}^{2}} = 0 \qquad Eq . ( S7 )$$

In this situation, the concentration of phages is determined by the Laplace equation—right term in Eq. (S7). Integrating this differential equation and applying the boundary conditions, ϕ(0) and ϕ(H), give us the concentration profile:

$$φ ( x ) = {φ}_{0} ( 1 - \frac{x}{H} ) \qquad Eq . ( S8 )$$

Applying this profile into the Fick's law equation, Eq. (S4), we obtain the general expression of the flux of phages in the leaky-gut model:

$${J}_{w} = \frac{{D}_{w} {0}_{0}}{H} \approx 1.4 \times {10}^{- 3} μ {m}^{- 2} {s}^{- 1} \qquad Eq . ( S9 )$$

Applying the value of the phage diffusion in water at body temperature, D_{w} (Eq. S3), the concentration of phage in the large intestine, ϕ₀=ϕₗᵢ (Table S5), and the height of the epithelial cell, H (Table S5), we obtain a flux of phages of 1.4×10⁻³ per unit area (μm²) and time (s).

How does this flux compare to the number of phages penetrating the body by the transcytosed mechanism? To answer this, we estimate the effective section of the channel (or number of epithelial cells removed) necessary to lead to the same number of phages per day obtained in the gut in Eq. (S3). This condition is expressed as:

J_{w}τS*_{w}=Φ_{tr,m}^{day} Eq. (S10)

That is, the flux times the time (τ=24 h) times the damaged surface (S*_{w}) equates the number of phages transcytosed per day in the long intestine (Eq. S3). This leads to:

$${S}_{w}^{*} = \frac{{Φ}_{tr , m}^{day}}{{J}_{w} τ} \approx 256.0 {mm}^{2} \qquad Eq . ( S11 )$$

Taking into account the section of an epithelial cell, S_{ec} (Table S6), we obtain that the number of damaged epithelial cells:

$${N}_{w}^{*} = \frac{{S}_{w}^{*}}{{S}_{ec}} \approx 10.24 \times {10}^{6} \qquad Eq . ( S11 )$$

Thus, the leaky-gut mechanism requires more than ten million epithelial cells to be removed to reach a similar number of phages penetrating the body compared to the phage transcytosis mechanism. Notice that the flux in this case was an upper limit, so the number of damaged cells is a lower limit. If we introduce more realistic details in the model (e.g., wall effects, non-homogeneous flux across the section, entropic cost to enter the channel), this number would increase considerably.

### Results

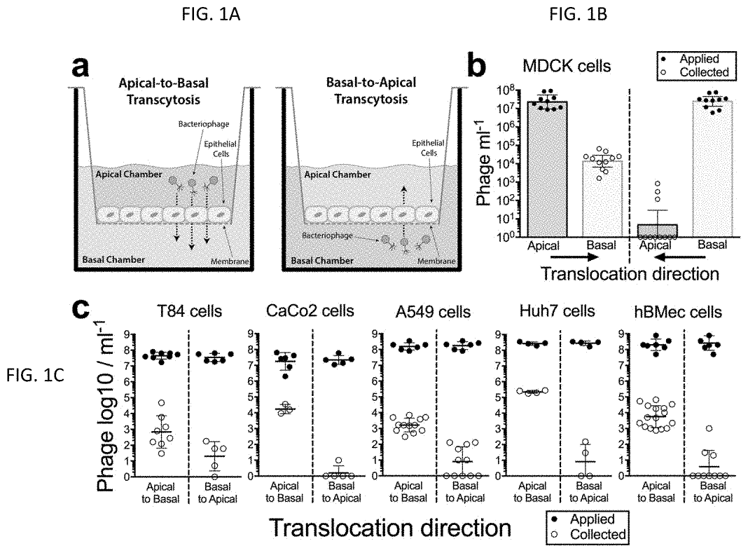

T4 phage transcytosis across eukaryotic cells. The directional transcytosis of T4 phage particles across eukaryotic cells was measured using Transwell inserts seeded with Madin-Darby canine kidney cells (MDCK) that were grown to confluence (FIG. 1A). All cells were cultured as high resistance monolayers to ensure transcytosis across the cell layer, rather than paracellular transport. Average transepithelial resistance (TER) measures were between 150-200 Ω*cm² and post-assay confluency was confirmed using Evans blue dye (Supplement, Fig S1). Phages were applied to either the apical or basolateral (basal) side of the cell layer at a mean concentration of 3.2×10⁷ phage ml⁻¹ and functionally translocated phages were collected in the contralateral chamber two hours later. Apical-to-basal transcytosis ranged from 3.6×10³ to 6.6×10⁴ phage ml⁻¹ (FIG. 1B, 1.95±1.94×10⁴ median±standard deviation, s.d., n=10, coefficient of variation, CV=83%) and basal-to-apical transcytosis ranged from 0 to 8.3×10² phage ml⁻¹ (125±267 median±s.d., n=10, CV=212%). This represented 0.1% and 0.0008% of the total phage applied being functionally transcytosed in the apical-to-basal and basal-to-apical direction, respectively (Table S1). Phage transcytosis across confluent cell layers had a significant preferential directionality for apical-to-basal transport (FIG. 1B, Mann-Whitney, n₁=n₂=10, U=0, P<0.0001 two tailed).

To determine whether these observations with the MDCK cell line could be extended to other human associated tissues, we examined cell lines derived from distinct organs, and which form confluent monolayers, including those from the gut (T84 and CaCo2), lung (A549), liver (Huh7) and brain (hBMec) (FIG. 1C). In each case, bidirectional transcytosis was observed with a preferential vector of dominance as shown by significant apical-to-basal directionality for all cell types (Table S2). The transcytosis of T4 phages across these diverse cell types is consistent with a generalized mechanism of phage transport across polarized epithelial cell monolayers with strong apical-to-basal transport directionality.

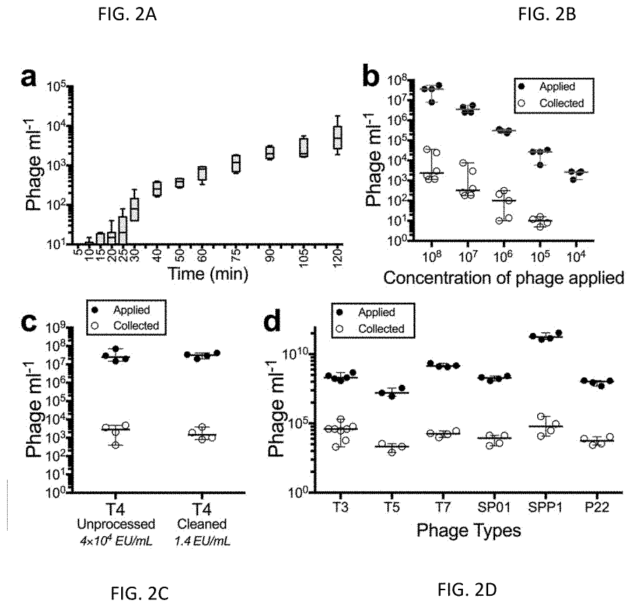

Functionality of phage transcytosis. The ingress of phages throughout the body has been previously described (4-12). However, there have been no quantitative measurements of the rate, dose or generality of the phenomenon. Using T4 phages and MDCK cells, the rate of apical-to-basal transcytosis was recorded over a two-hour period (FIG. 2A). Phage were detected within the basal chamber as early as ten minutes after application, with consistent transport occurring within 30 min and steadily increasing up to two hours. The rate of phage transcytosis was 0.325 μm/h, as calculated by per unit time (h), surface area (μm²) and applied concentration (phage ml⁻¹). Apical-to-basal transcytosis was dose-dependent (FIG. 2B). As the dose of apically applied phage was sequentially reduced by ten-fold, the basal collection sequentially decreased in a proportional manner, continuing down until a dosage of 10⁴ phage ml⁻¹, which represented the limit of detection for the assay.

Phage preparations are often contaminated by host bacteria macromolecules, with the major pyrogen being lipopolysaccharide (endotoxin) (32). Endotoxin is known to elicit a wide range of pathophysiological effects in the body, stimulating cellular and immune responses (33). To investigate whether residual endotoxins were triggering phage transcytosis, we compared a T4 phage stocks before and after removal of endotoxins (34). The removal of endotoxins produced no significant change in apical-to-basal transcytosis of T4 phage (FIG. 2C, Mann-Whitney two tailed, n₁=n₂=4, U=6, P=0.6857).

The generality of phage transcytosis was next tested using diverse phages across the order of Caudovirales, encompassing phages from; the three major morphotypes (Myoviridae, Siphoviridae, Podovirdae), Gram positive and negative bacterial hosts, and phages originating from soil and intestinal reservoirs. All phages tested elicited strong apical-to-basal transcytosis (FIG. 2D).

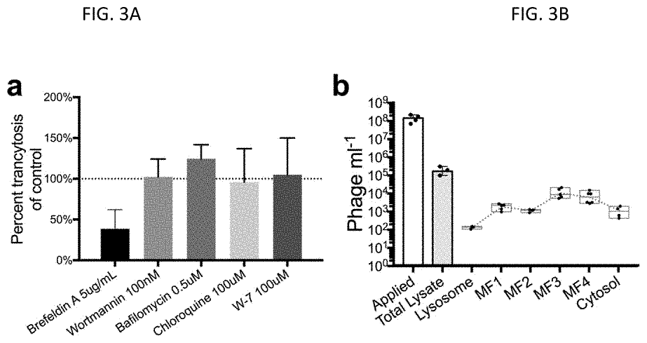

Permeation of phages throughout the eukaryotic cell. The mechanism of phage access to Eukaryotic cells remains ambiguous (22, 24, 29). To identify this mechanism, we applied chemical inhibitors known to arrest steps along the transcytotic pathway in MDCK cells prior to application of T4 phages. Inhibition of phage transcytosis was reported as the percentage of phages transcytosed across inhibitor-treated cells, compared to cells treated with a solvent control (FIG. 3A and Table S3). Treatment with Brefeldin A, which inhibits protein translocation between the endoplasmic reticulum and the Golgi apparatus (35), showed significant but incomplete inhibition of transcytosis, with 38.5% of phages transcytosed compared to a solvent control. We observed no significant effects of wortmannin (an inhibitor of phostadidylinositol-3-kinase and receptor mediated transcytosis), bafilomycin (an inhibitor of endosomal acidification), chloroquine (an inhibitory of endosomal acidification) and W-7 (an antagonist of calmodulin that inhibits microtubule endocytic membrane transport). This suggests that phages transit through the Golgi apparatus, which is in agreement with previous observations of phages within the Golgi region (29), before being exocytosed via the basolateral membrane.

The proportion of fluorescence-positive cells treated with BFA and labeled T4 phages was 9.98% (n=1008, 9.98%±0.78%, mean±s.e.)

Subcellular fractionations were performed to assess T4 phage localization within MDCK cells. Cells were treated with T4 phage for either 5 minutes or 18 hours, washed, fractionated and the vesicular and cytoplasmic cellular components collected. Cells treated for 5 minutes showed no detectable phages in any subcellular fractions, likely due to the insufficient incubation time (Fig S2). In comparison, cells treated for 18 hours showed 0.14% of total phage applied being functionally accumulated within the total cell lysate (FIG. 3B, 1.7±0.91×10⁵, median±s.d., n=4, CV=49%). Following fractionation, phages were detected in all major subcellular compartments of the cell including the cytosol, lysosomes, and were enriched within the denser microsomal fractions (MF) typically associated with the Golgi apparatus and endoplasmic reticulum (36) (Table S4).

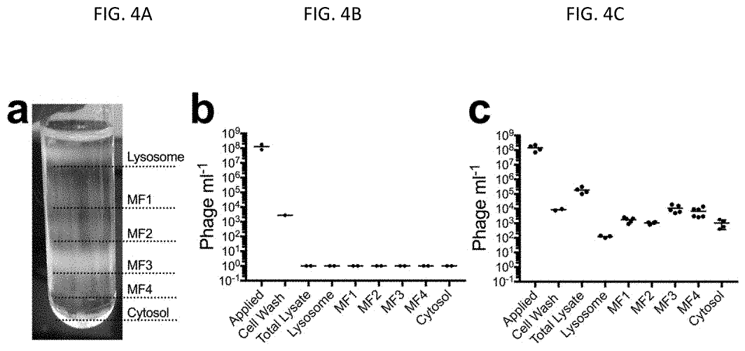

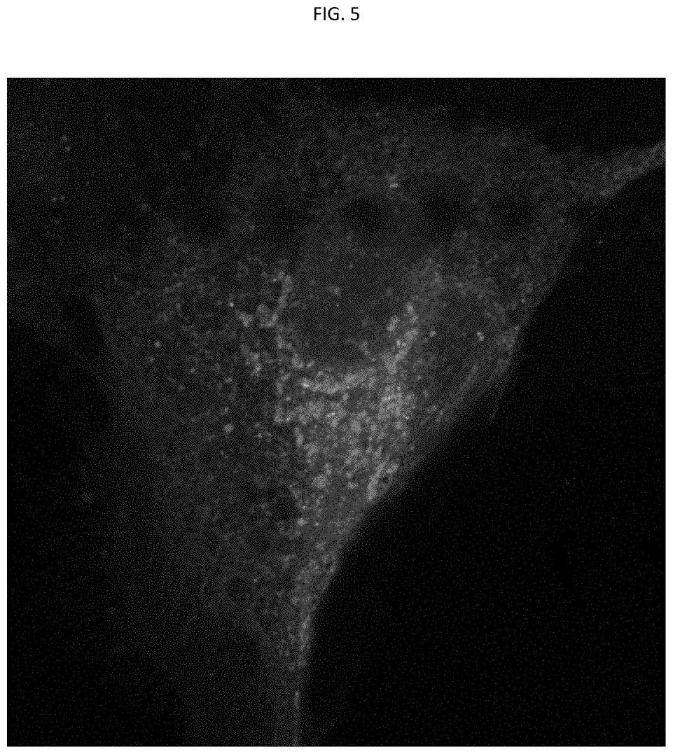

T4 phage were fluorescently labeled and visualized within MDCK cells (FIG. 4A-C). Fluorescent particles were visualized as both discrete puncta and diffuse clouds within the cytoplasm (Fig S3).

The proportion of fluorescence-positive cells treated with labeled T4 phages was 10.54% (n=2961, 10.54%±0.81%, mean±s.e.) and 1.7% (n=1650, 1.7%±0.5%, mean±s.e.) as analyzed by epifluorescence microscopy and flow-cytometry, respectively. This is in contrast to our prior observation that 0.1% of total phage applied were transcytosed (FIG. 1B) and 0.14% accumulated within the cell (FIG. 3B). Suggesting a small fraction of cells are responsible for the transcytosis and subsequent intracellular accumulation of phages. Differences are likely due to the detection of functional phages in transcytosis and cellular assays versus detection of fluorescence-positive cells, that may contain either functional or inactivated phages, in fluorescence-based assays. Results suggests that between 1-10% of endocytosed phages are functionally transcytosed across the cell with the remaining phages being inactivated or persisting within the cell.

Estimates of phage ingress to the human body. Within the human body the largest aggregation of phages resides within the gut (17, 37). Although the concentration of bacteria within the human gut (averages 9.17×10¹⁰ per gram of feces) has been well documented (38, 39), direct quantification of phages is comparatively lacking. Based on three literature references utilizing direct counts and DNA yield, we estimate 5.09×10⁹ phage per gram of feces (2, 40, 41), yielding 2.09×10¹² phage within the colon of an average human (39). Using our experimentally derived rate of phage transcytosis across T84 gut epithelial cells, and assuming a 4.4-fold increased concentration of phage in mucosal surfaces (42), we estimate that the average human body transcytoses 3.09×10¹⁰ phages per day. Finally, we contrast this estimate with a competing mechanism of access to the body via a ‘leaky gut’. In this model free phages are allowed to bypass confluent epithelial layers at sites of damage and inflammation, gaining access to the body directly (19, 20). To achieve similar phage ingress to our transcytosis model, we estimate this requires lesions of approximately 256 mm² within the gastrointestinal tract, or the removal of 10.24×10⁶ epithelial cells. This amount of intestinal damage would likely result in significant inflammation of the gut and is in contrast to the detection of phages in asymptomatic humans (8-12).

FIG. 1A-C illustrates transcytosis of bacteriophages occurs in a preferential apical-to-basal direction across diverse cell layers, a) Experimental system to investigate phage transcytosis. Phage T4 were applied to either the apical or basolateral (basal) cell chambers, incubated for two hours at 37° C. and transcytosed phages were sampled and quantified in the contralateral chamber. b) Transcytosis of T4 phages across Madin-Darby canine kidney cells (MDCK) in either an apical-basal or basal-apical direction. c) Transcytosis of T4 phages across T84 cells (colon epithelial), CaCo2 cells (colon epithelial), A549 cells (lung epithelial), Huh7 (hepatocyte epithelial-like) and hBMec cells (brain endothelial). Scatter plots show median, error bars represent 95% confidence interval, each point represents the average of three technical replicates.

FIG. 2A-2D illustrates rate and diversity of apical-to-basal phage transcytosis. a) The rate of T4 phage transcytosis across MDCK cells over a two-hour period, b) Transcytosis of T4 phage applied to MDCK cells at sequentially decreasing log 10 concentrations. c) Transcytosis of unprocessed (4×10⁴ EU ml⁻¹) and cleaned (1.4 EU ml⁻¹) T4 phages across MDCK cells. d) Transcytosis of diverse phage types across MDCK cells. Bar plot shows mean, error bars show min-max values. Scatter plots show median, error bars represent 95% confidence interval, each point represents the average of three technical replicates.

FIG. 3A-3B illustrates inhibition of phage transcytosis and subcellular localization, a) Percent transcytosis of T4 phages across MDCK cells pretreated with chemical inhibitors compared to a solvent control. b) Fractionation of MDCK cells treated with T4 phages for 18 hrs. Cells were washed, lysed and total number of phage in cell lysates quantified (Total Lysate). Total lysates were then fractionated using Lysosome Enrichment Kit and six cellular fractions were collected. Lysosome and microsomal fractions (MF) were pelleted, washed, lysed and collected, cytosol fractions were collected and phage quantified in all collected fractions by bacterial plating. Bar plot shows mean, error bars show standard deviation. Scatter plots show median, error bars represent 95% confidence interval, each point represents the average of three technical replicates.

FIG. 4A-4C illustrates subcellular fractionation of MDCK cells treated with T4 phages, a) Fractionation of cell lysate showing the six cellular fractions collected. b) 5 min treatment of MDCK cells showing no phages present in any cellular fractions. Phages were detected in the cell washes before fractionation. c) 18 hour treatment of MDCK cells showing phages present in all major cellular fractions.

FIG. 5 illustrates a visualization of intracellular phages. Fluorescence-labeled T4 phages (green) were transcytosed across MDCK cells stained with DAPI (blue), golgi (red) and plasma membranes (white). Images show fluorescent phages particles associated with subcellular compartments of the cell.

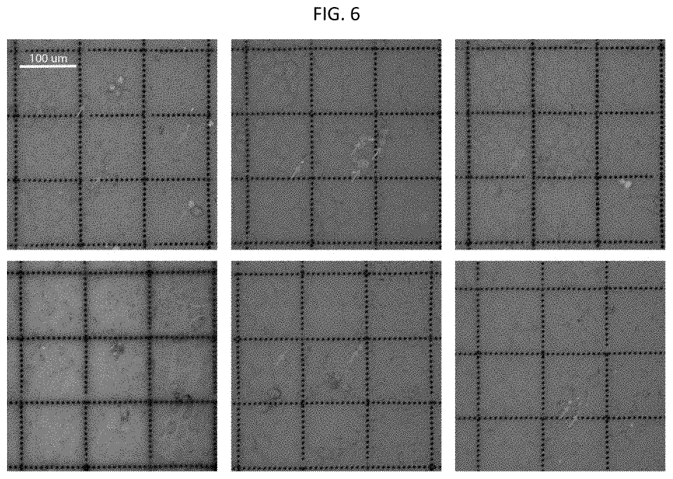

FIG. 6, illustrates images of MDCK cells treated with SYBR-Gold labeled T4 phages grown on Ibidi μ-Slide CorrSight™ Live. Red arrows show cells containing distinct SYBR-labeled puncta, indicating uptake of phage particles in microsomal vesicles. Blue arrows show cells with diffuse SYBR-labeled cytoplasm, indicating release of phage or labeled-phage DNA into the cytoplasm. Images were imaged with DIC and fluorescence at 495 nm excitation with 540 nm emission, scale bar=100 μm.

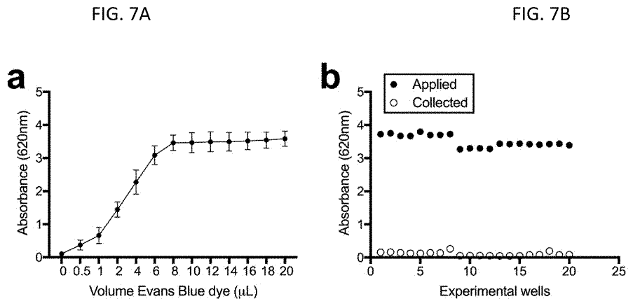

FIG. 7A-7B illustrates a post-assay confluency test with Evans blue dye: a) Absorbance of Evans blue standard curve; b) Post-assay Evans blue dye concentrations from applied and collected wells, showing minimal leakage or paracellular transport occurred in experimental assays.

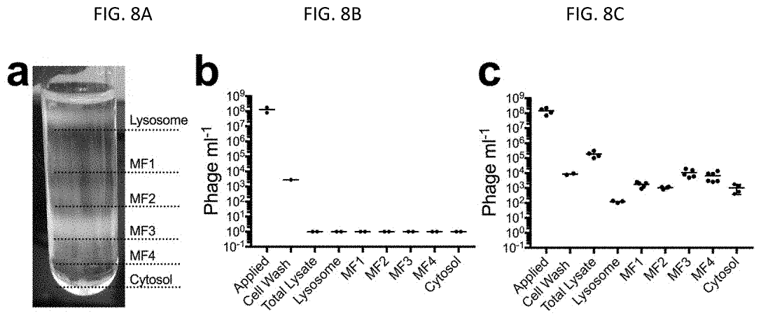

FIG. 8A-8C illustrates subcellular fractionation of MDCK cells treated with T4 phages: a) Fractionation of cell lysate showing the six cellular fractions collected; b) 5 min treatment of MDCK cells showing no phages present in any cellular fractions, phages were detected in the cell washes before fractionation; c) 18 hour treatment of MDCK cells showing phages present in all major cellular fractions.

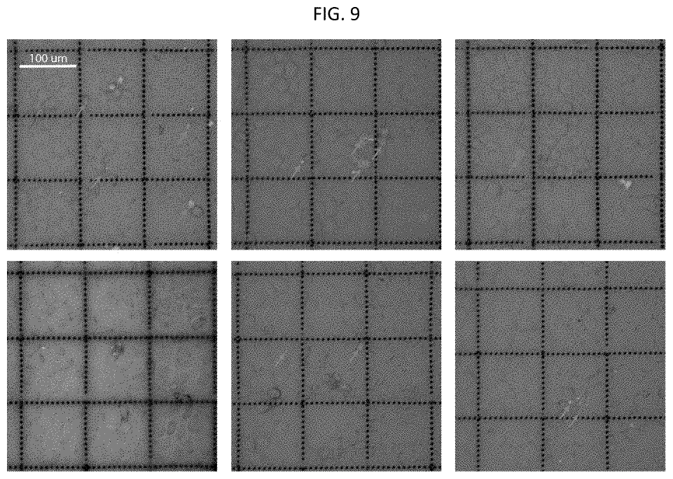

FIG. 9 illustrates images of MDCK cells treated with SYBR-Gold labeled T4 phages grown on Ibidi μ-Slide CorrSight™ Live. Red arrows indicated cells containing distinct SYBR-labeled puncta, blue arrows indicate cells with diffuse SYBR-labeled cytoplasm. Images were imaged with DIC and fluorescence at 495 nm excitation with 540 nm emission, scale bar=100 μm.

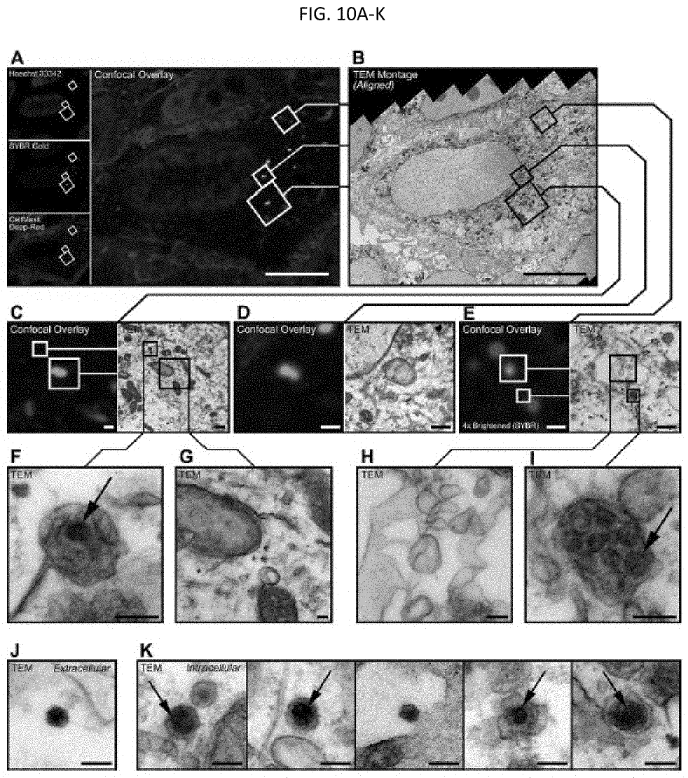

We selected a SYBR-gold positive target cell using confocal microscopy (FIG. 10A) and processed the cell for ultrastructure inspection of intracellular phage particles using correlative light electron microscopy (CLEM; FIG. 10B), see e.g., Padman et al 2014, An Improved Procedure for Subcellular Spatial Alignment during Live-Cell CLEM, PLoS One 9:e95967. Membrane-bound virus-like particles were visible within the target cell using transmission electron microscopy (TEM), however these particles did not colocalize with SYBR-gold fluorescence, and conversely, fluorescent signal was found in vesicles that did not appear to contain T4 phages (FIG. 10C-I). The lack of colocalization between SYBR-gold fluorescence and virus-like particle ultrastructure may be attributed to numerous factors, including but not limited to; insufficient fluorescent labeling of T4 phages, limited fluorescence detection of individual labeled-phages, pH instability of SYBR-gold stain (pH 7-8.5) under the acidic conditions found within the endosomal lumen (pH 5-6.2) (see, e.g., Scott C C, Gruenberg J, 2011, Ion flux and the function of endosomes and lysosomes: pH is just the start, BioEssays 33:103-110), or the degradation of labeled phages within the cell. Despite the lack of colocalization, both extracellular and intracellular virus-like particles were found within the SYBR-positive target cell using TEM (FIGS. 10F, I, J & K). Internalized virus-like particles were visualized as electron-dense, icosahedral structures of less than 100 nm, within membrane-bound compartments; suggesting that phages are transcytosed via the endomembrane system.

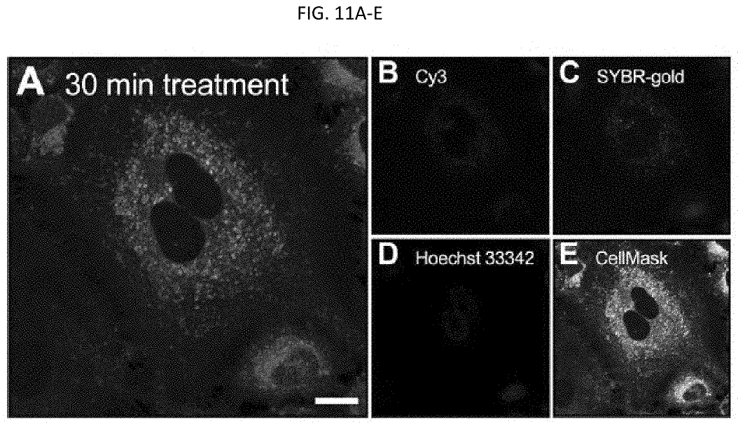

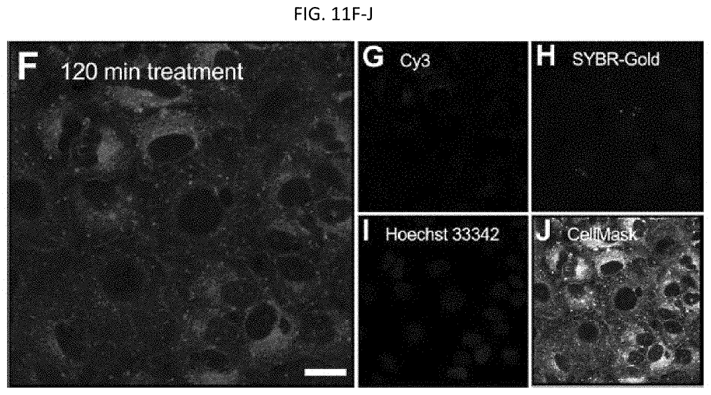

To address the lack of fluorescence and TEM ultrastructure correlation, we performed a time-series experiment using dual-fluorescence labeled phages that were incubated with MDCK cells for either 30 min or two hrs (FIG. 11). Phages were labeled with both the DNA-complexing SYBR-gold stain and a capsid-linked Cy3 stain, followed by imaging using confocal microscopy. Cellular incubations at 30 min revealed correlation between SYBR-gold and Cy3 fluorescence within membrane-bound vesicles of the cell (FIGS. 11A, B, C & E). Comparatively, cellular incubations for two hrs (same incubation time used for CLEM experiment) showed evidence of disassociation of dual fluorescence signals within the cell, with distinct SYBR-gold and Cy3-positive vesicles observed (FIG. 11F-H). This disassociation of dual-labeled, fluorescence signals suggests either an instability of fluorescence dyes or the degradation of phage internalized phage particles occurred, and may explain the lack of signal colocalization in the previous CLEM results (FIG. 10). Further work using real-time microscopy approaches, labeling of additional endomembrane compartments, and repeat CLEM analyses using dual-labeled phages are required to further elucidate how phage transcytosis occurs.

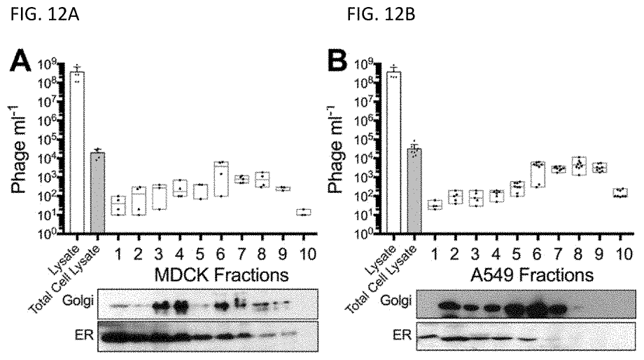

Permeation and inhibition of phages throughout the eukaryotic cell: Subcellular fractionation was performed to assess intracellular T4 phage dispersal within MDCK and A549 cells. To ensure maximal uptake and penetration of phages throughout the subcellular structure, cells were incubated with phages for 18 hrs, extensively washed, fractionated and the vesicular and cytoplasmic cellular components collected. Vesicular fractions were then split, with half of the fraction lysed using chloroform and the total number of phage quantified by plating with their bacterial host, and the remaining fraction protein precipitated and analyzed by immunoblotting using Golgi and endoplasmic reticulum markers (FIG. 12). Phages accumulated within the total cell lysate (MDCK; 2±1×10⁴, median±s.d., n=5, CV=53%, A549; 2.6±2.3×10⁴, median±s.d., n=9, CV=70%.) and were detected in all subcellular fractions of the cell. Intracellular phages were found to be enriched within the denser endomembrane fractions of the cell that were associated with the Golgi apparatus.

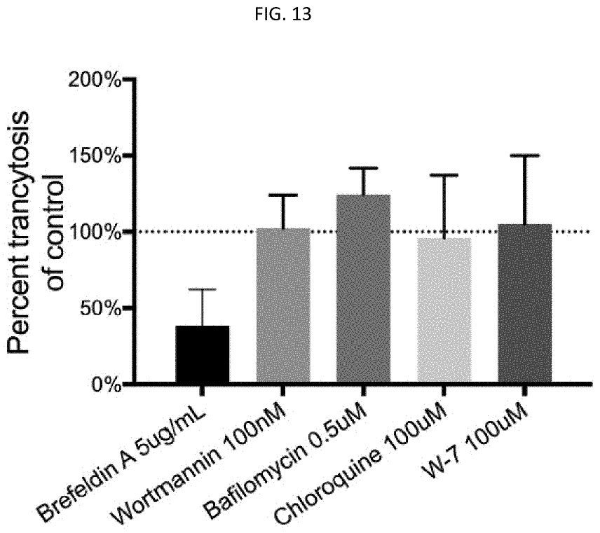

Before this invention, the mechanism of phage transcytosis across eukaryotic cells remained ambiguous, see e.g., Duerkop et al., 2013, Resident viruses and their interactions with the immune system, Nat Immunol 14:654-9; Merril, et al., 1996, Long-circulating bacteriophage as antibacterial agents, Proc Natl Acad Sci 93:3188-3192; Aronow, et al., 1964, Electron microscopy of in vitro endocytosis of T2 phage by cells from rabbit perioneal exudate, J Exp Med 120. We applied chemical inhibitors known to arrest steps along the transcytotic pathway to MDCK cells 18 hrs prior to application of T4 phages. Inhibition of phage transcytosis was reported as the percentage of phages transcytosed across inhibitor-treated cells compared to cells treated with a solvent control (FIG. 13). Treatment with brefeldin A, which inhibits post-Golgi membrane traffic and protein translocation between the endoplasmic reticulum and the Golgi apparatus, showed significant but incomplete inhibition of transcytosis, with 38.5% of phages transcytosed compared to a solvent control. We observed no significant treatment effects of wortmannin (an inhibitor of phostadidylinositol-3-kinase and receptor mediated transcytosis), bafilomycin (an inhibitor of endosomal acidification), chloroquine (an inhibitory of endosomal acidification) or W-7 (an antagonist of calmodulin that inhibits microtubule endocytic membrane transport). This suggests that phages may transit through the Golgi apparatus before being exocytosed via the basolateral membrane. However, these inhibitors can impact cellular trafficking in a range of ways and further direct evidence is needed to confirm this.

Estimates of phage ingress to the human body: Within the human body the largest aggregation of phages resides within the gut (20, 43). Although the concentration of bacteria within the human gut (averages 9.17×10¹⁰ per gram of feces) has been well documented (44, 45), direct quantification of phages is comparatively lacking. Based on three literature references (Clokie et al., 2011. Phages in nature. Bacteriophage 1:31-45; Kim et al., 2011, Diversity and abundance of single-stranded DNA viruses in human feces. Appl Environ Microbiol 77:8062-70; Reyes et al., 2013, Gnotobiotic mouse model of phage-bacterial host dynamics in the human gut, Proc Natl Acad Sci USA 110:20236-41) utilizing direct counts and DNA yield, we estimate 5.09×10¹ phage particles per gram of feces, yielding 2.09×10¹² phage particles within the colon of an average human. Using our experimentally derived rate of phage transcytosis across T84 gut epithelial cells, and assuming a 4.4-fold increased concentration of phage in mucosal surfaces, we estimate that the average human body transcytoses 3.1×10¹⁰ phages per day.

Finally, we contrast this estimate with a competing mechanism of access to the body via a ‘leaky gut’. In this model, free phages are allowed to bypass confluent epithelial layers at sites of damage and inflammation, gaining access to the body directly. To achieve similar phage ingress to our transcytosis model, we estimate this requires lesions of approximately 256 mm² within the gastrointestinal tract, or the removal of 10.24×10⁶ epithelial cells. This amount of intestinal damage would likely result in significant inflammation of the gut and is in contrast to the detection of phages within the blood and serum of asymptomatic humans.

Cellular investigations showed phages were capable of accessing all subcellular fractions of the eukaryotic cell (FIG. 10) with intra-cellular transport suggested to traffic through the Golgi apparatus (FIG. 11). The strong apical-to-basal transport suggests that epithelial cells are preferentially transporting phages in this direction. Based on these results we estimate that thirty-one billion phage transcytotic events occur within the average human body per day, while comparable ingress via ‘leaky gut’ is estimated to require significant damage and inflammation to the gastrointestinal tract, see e.g., Handley et al., 2012, Pathogenic simian immunodeficiency virus infection is associated with expansion of the enteric virome. Cell 151:253-66; Karimi, et al., 2016, Bacteriophages and phage-inspired nanocarriers for targeted delivery of therapeutic cargos. Adv Drug Deliv Rev 106:45-62.

### DISCUSSION

The observation of microorganisms present within the blood and body is long-standing. Numerous mechanisms for bacterial uptake and invasion, and their subsequent effects have been identified. Comparatively, the mechanisms for bacteriophage uptake and potential effects on the body remain unaddressed. This is partly due to an underappreciated notion for phage-Eukaryote interactions, but also stems from the irregular and low rate of phage transcytosis, and the inherent difficulties associated with the molecular identification of low abundance phage (45). The identification of an intra-body ‘phageome’ presents a significant challenge (46), yet the potential implications for health and disease demands additional research into this underexplored area.

The transcytosis of bacteriophage across epithelial cells (FIG. 1) provides a mechanistic explanation for the occurrence of phage within the human body in the absence of disease (6-10). Apical-to-basal transcytosis was observed with every phage type investigated across diverse cell lines (FIG. 1C. 2D). Inhibition of phage transcytosis with brefeldin A (FIGS. 3A & 4) suggests the intra-cellular transport of phages likely involves the Golgi apparatus. Cellular analysis revealed phages were capable of accessing most subcellular compartments (FIG. 3C), with paracellular transport across an intact epithelial barrier not likely to be a significant mechanism. The strong apical-to-basal transport suggests that epithelial cells are preferentially transporting phages into the body. We estimate thirty-one billion phage transcytotic events occur within the average human body per day, while comparable ingress via ‘leaky gut’ is estimated to require significant damage and inflammation to the gastrointestinal tract (19, 20).

If the human body is perpetually absorbing phage, what might be the intended function? The major reservoir of phage in the body is observed in the gastrointestinal tract. Over the lifetime of a human these gut phage co-evolved with the microbiome, and represent the most genetic diversity and “biological dark matter” in the body (47, 48). At the simplest level, the presence of a low-level but continuous stream of phage originating from the gut and disseminating through the blood, lymph and organs, may provide the host with a system-wide antimicrobial against the intrusion of any opportunistic gut microbe. Their dissemination may have additional roles in cellular disease, cancer recognition and even the vertical transmission of adapted gut phage populations from mother to infant through breast milk (49-51).

At the same time, this continuous and low-level stream of phage represents a persistent influx of foreign and thus immunogenic particles throughout the body. Phages capacity to stimulate humoral responses and induce anti-phage antibodies is dependent on both their route of administration and dosage (7, 52). Transcytosed phage are continuously dosed to the body at relatively low levels, with a diversity that reflects current gut conditions and that lack costimulatory signals such as endotoxins (32). As such the immunostimulatory effects of transcytosed phages on the body are largely unknown. Nonetheless, their presence within the body could provide long-term immunological tolerance through interactions with regulatory T cell populations (53). Alternatively, aberrant transcytosis may contribute to enhanced immune responses, allergic reactions and inflammatory diseases (54).

Perhaps the greatest potential function of transcytosed gut phages is the utilization of their astounding genetic diversity by the body directly. Previous work using recombinant T4 phages has already documented the delivery and expression of single or multiple genes to Eukaryotic cells both in vitro and in vivo (25). The transcytosis of diverse phages reported here provides a mechanism to traverse the Eukaryotic cell. The subsequent intracellular dissemination of these phages and their genetic material provides a means to directly affect the Eukaryotic cell. Nominally this allows for horizontal gene transfer between phages and Eukaryotes (55) and the direct uptake and expression of phage genetic material within the body, potentially representing an unexplored third external genome (56). Studies provided herein demonstrate that the transcytosis of bacteriophage into the body across polarized epithelial cells is a naturally occurring and ubiquitous process, and for the first time validates the use and application of phages in an in vivo biomedical setting.

TABLE S1
Directional transcytosis of T4 phages
across confluent MDCK monolayers.

| Apical-to-basal transcytosis | | | Basal-to-apical transcytosis | | |
|---|---|---|---|---|---|
| | Collect- | Percent- | | Collect- | |
| Applied | ed | age | Applied | ed | Percentage |
| 1.1 × 10⁷ | 5 × 10³ | 0.05% | 6 × 10⁶ | 3 × 10² | 0.005% |
| 1.8 × 10⁷ | 1.9 × 10⁴ | 0.1% | 1.2 × 10⁷ | 0 | 0% |
| 1.2 × 10⁷ | 3.7 × 10³ | 0.03% | 8 × 10⁶ | 0 | 0% |
| 2.5 × 10⁷ | 1.2 × 10⁴ | 0.05% | 2.8 × 10⁷ | 0 | 0% |
| 8.7 × 10⁶ | 1.3 × 10⁴ | 0.14% | 2.7 × 10⁷ | 1.2 × 10² | 0.0004% |
| 3.8 × 10⁷ | 6.6 × 10⁴ | 0.17% | 7.6 × 10⁷ | 0 | 0% |
| 9.2 × 10⁶ | 2.5 × 10⁴ | 0.27% | 7.2 × 10⁷ | 0 | 0% |
| 3.3 × 10⁷ | 4.8 × 10⁴ | 0.15% | 3.1 × 10⁷ | 0 | 0% |
| 9.2 × 10⁷ | 2 × 10⁴ | 0.02% | 3.5 × 10⁷ | 0 | 0% |
| 8.6 × 10⁷ | 2.5 × 10⁴ | 0.03% | 3.2. × 10⁷ | 8.3 × 10² | 0.0026% |

TABLE S2
Collected transcytosis of T4 phages across confluent epithelial monolayers.

| | | | | | | | Mann-Whitney | | Paired | | |
|---|---|---|---|---|---|---|---|---|---|---|---|
| | | | | | | | two tailed | | t-test | | |
| | Apical-to-basal transcytosis | | | Basal-to-apical transcytosis | | | Non-parametric | | Parametric | | |
| Cells | Median ± s.d. | n | CV | Median ± s.d. | n | CV | U | P | t | df | P |
| MDCK | 1.95 ± 1.94 × 10⁴ | 11 | 90% | 1 ± 267 | 11 | 224% | 0 | <0.0001 | 3.8 | 9 | 0.0042 |
| T84 | .79 ± 1.74 × 10⁴ | 7 | 218% | 60 ± 72 | 5 | 118% | 2 | 0.008 | 2.3 | 3 | 0.1031 |
| CaCo2 | 1.6 ± 1.3 × 10⁴ | 3 | 67% | 1 ± 4 | 5 | 143% | 0 | 0.0179 | 2.6 | 2 | 0.1231 |
| A549 | 1.6 ± 2.2 × 10³ | 12 | 89% | 10 ± 54 | 11 | 137% | 0 | <0.0001 | 3.6 | 10 | 0.0046 |
| Huh7 | 2.3 ± .47 × 10⁵ | 4 | 20% | 15 ± 71 | 4 | 156% | 0 | 0.0286 | 3 | 9 | 0.0148 |
| hBMec | .48 ± 2 × 10⁴ | 16 | 131% | 1 ± 314 | 10 | 297% | 3 | <0.0001 | 9.7 | 3 | 0.0024 |

TABLE S3
Inhibition of T4 phages transcytosis across confluent MDCK monolayers by chemical inhibitors.

| | | | | | | | | | One-way | | | |
|---|---|---|---|---|---|---|---|---|---|---|---|---|
| | | | | | | | | | ANOVA | | One-way | |
| | | | | | | | | | Non-parametric, | | ANOVA | |
| Solvent transcytosis | | | Inhibitors | | | Inhibitor transcytosis | | | Kruskal-Wallis | | Parametric | |
| Median ± s.d. | n | CV | Inhibitor | Conc. | | Median ± s.d. | n | CV | Mean rank diff. | P | Mean diff. | P |
| 7.9 ± 3 × 10⁴ | 40 | 41% | Brefeldin A | 5 | μgmL⁻¹ | 2.3 ± 2.2 × 10⁴ | 8 | 73% | 27.36 | 0.003 | 4.4 × 10⁴ | 0.0004 |
| 7.9 ± 3 × 10⁴ | 40 | 41% | Wortmannin | 100 | nM | 8.1 ± 2.9 × 10⁴ | 8 | 36% | −5.14 | >0.99 | −4.5 × 10³ | 0.99 |
| 7.9 ± 3 × 10⁴ | 40 | 41% | Bafilomycin | 0.5 | μM | 1 ± .19 × 10⁵ | 3 | 21% | −13.58 | >0.99 | −1.9 × 10⁴ | 0.67 |
| 7.9 ± 3 × 10⁴ | 40 | 41% | Chloroquine | 100 | μM | 5.3 ± 2.7 × 10⁴ | 4 | 42% | 4.93 | >0.99 | 1.1 × 10⁴ | 0.90 |
| 7.9 ± 3 × 10⁴ | 40 | 41% | W-7 | 100 | μM | 8.2 ± 1.1 × 10⁴ | 8 | 14% | −6.95 | >0.99 | 6.6 × 10³ | 0.95 |

TABLE S4
Subcellular fractionation of MDCK cells treated
with T4 phage for 5 min and 18 hrs.

| | 5 m treatment | | | 18 hr treatment | | |
|---|---|---|---|---|---|---|
| Fraction | Median ± s.d. | n | CV | Median ± s.d. | n | CV |
| Applied | 1.3 ± .7 × 10⁸ | 2 | 54% | 1.5 ± .67 × 10⁸ | 4 | 46% |
| Cell Wash | 2.8 ± .2 × 10³ | 2 | 10% | 8.5 ± 1.2 × 10³ | 2 | 14% |
| Cell Lysate | 0 ± 0 | 2 | 0% | 1.7 ± .91 × 10⁵ | 4 | 49% |
| Lysosome | 0 ± 0 | 2 | 0% | 130 ± 20 | 3 | 17% |
| MF1 | 0 ± 0 | 2 | 0% | 2 ± .64 × 10³ | 5 | 37% |
| MF2 | 0 ± 0 | 2 | 0% | 1.1 ± .18 × 10³ | 4 | 17% |
| MF3 | 0 ± 0 | 2 | 0% | 8 ± 6.2 × 10³ | 5 | 59% |
| MF4 | 0 ± 0 | 2 | 0% | 5.9 ± 4.6 × 10³ | 6 | 69% |
| Cytosol | 0 ± 0 | 2 | 0% | 950 ± 656 × 10³ | 4 | 64% |

TABLE S5
Physiological parameters associated with the large intestine.

| Description | Parameter | Value | | Reference |
|---|---|---|---|---|
| Surface area (large intestine) | Sₗᵢ | 3,460 | cm⁻² | Snyder et al., 1975 - (5) |
| Volume (large intestine) | Vₗᵢ | 409 | ml | Sender et al., 2016 - (6) |
| Number of epithelial cells | N_{ec} | 7.20 × 10¹¹ | | Snyder et al.. 1975 - (5) |
| Dimensions of epithelial cells | width × length × | 5 μm × 5 μm × 40 μm | | Snyder et al., 1975 - (5) |
| | height | | | |
| Surface area (epithelial cell) | S_{ec} | 25 | μm² | Snyder et al., 1975 - (5) |
| Number of phages in colon (in 0.41 | Φₗᵢ | 2.09 × 10¹² | | Sender et al., 2016 - (6) |
| litres of large intestine) | | | | |
| Concentration of phages in colon | ϕₗᵢ | 5.11 × 10⁹ | ml⁻¹ | ϕₗᵢ = Φₗᵢ/Vₗᵢ |

TABLE S6
Experimental parameters of the transcytosis assay.

| Description | Parameter | Value | |
|---|---|---|---|
| Volume of upper Transwell | Vᵤₚ | 250 | μl |
| Volume of lower Transwell | Vₗₒ | 250 | μl |
| Surface area of a single Transwell | Sₗₒ | 1.12 | cm⁻² |
| Time of incubation | tᵢₙ | 2 | h |
| Apical concentration of T4 | ϕᵤₚ | 4.70 × 10⁷ | ml⁻¹ |
| phages applied to T84 cells | | | |
| Basal concentration of T4 | ϕₗₒ | 6.99 × 10³ | ml⁻¹ |
| phages collected from T84 cells | | | |

TABLE S7
Parameters associated with the transcytosed
phages in humans model.

| Description | Parameter | Value | Equation |
|---|---|---|---|
| Phage transcytosis rate per | rₜᵣ | 0.166 μm/h | Eq. S1 |
| unit time, unit area (wall), and | | | |
| phage apical concentration | | | |
| Number of phage transcytosed | Φ^{day}ₜᵣ | 7.04 × 10⁹ | Eq. S2 |
| per day in humans | | phages/day | |
| Number of phage transcytosed | Φ^{day}_{tr, m} | 30.97 × 10⁹ | Eq. S3 |
| per day in humans (mucus factor) | | phages/day | |

TABLE S8
Parameters associated with the leaky gut model.

| Description | Parameter | Value | | Reference |
|---|---|---|---|---|
| Boltzmann constant | k_{B} | 1.38 × 10⁻²³ | K/J | — |
| Absolute temperature (body) | T | 310.15 | K | T = 273.15 + t, where t = 37° C. |
| Phage effective radius | R_{ϕ} | 30 | nm | Ackermann, 2007 - (7) |
| Water viscosity at body | v_{w} | 0.6913 | mPa s | IAPWS, 2008 - (8) |
| temperature | | | | |

### REFERENCES FOR ALL BUT METHODS

1. Rohwer F, Segall A M. 2015. Nature. 528(7580):46-48
2. Clokie M R, Millard A D, Letarov A V, Heaphy S. 2011. Bacteriophage. 1(1):31-45
3. Breitbart M. Hewson I, Felts B, Mahaffy J M, Nulton J, et al. 2003. J. Bacteriol. 185(20):6220-23
4. Dabrowska K, Switala-Jelen K, Opolski A, Weber-Dabrowska B, Gorski A. 2005. J. Appl. Microbiol. 98(1):7-13
5. Górski A, Wazna E, Dabrowska B-W, Dabrowska K, Switala-Jelen K, Miedzvbrodzki R. 2006. FEMS Immunol. Med. Microbiol. 46(3):313-19
6. Keller R, Engley F B. 1958. Exp. Biol. Med. 98(3):577-80
7. Slopek S, Weber-Dabrowska B, Dabrowski M, Kucharewicz-Krukowska A. 1987. Arch. Immunol. Ther. Exp. (Warsz). 35(5):569-83
8. Parent K, Wilson I D. 1971. Gut. 12(12):1019-20
9. Fancello L, Monteil S, Popgeorgiev N, Rivet R, Gouriet F, et al. 2014. PLoS One. 9(4):e93367
10. Breitbart M, Rohwer F. 2005. Biotechniques
11. Gaidelyte A, Vaara M, Bamford D H. 2007. PLoS One. 2(11):e1145
12. Weber-Dabrowska B. Dabrowski M, Slopek S. 1987. Arch. Immunol. Ther. Exp. (Warsz). 35(5):563-68
13. Dubos R, Straus J, Pierce C. 1943. J Exp Med. 78(3):161-68
14. Frenkel D, Solomon B. 2002. Proc. Natl. Acad. Sci. U.S.A 99(8):5675-79
15. Barrow P, Lovell M, Berchieri A. 1998. Clin. Diagn. Lab. Immunol. 5(3):294-98
16. Geier M R, Trigg M E, Merril C R. 1973. Nature. 246(5430):221-23
17. Reyes A, Haynes M, Hanson N, Angly F E, Heath A C, et al. 2010. Nature. 466(7304):334-38
18. Minot S, Bryson A, Chehoud C, Wu G D, Lewis J D, Bushman F D. 2013. Proc. Natl. Acad. Sci. 1300833110-
19. Handley S A, Thackray L B, Zhao G, Presti R, Miller A D, et al. 2012. Cell. 151(2):253-66
20. Karimi M, Mirshekari H, Moosavi Basri S M. Bahrami S. Moghoofei M, Hamblin M R. 2016. Adv. Drug Deliv. Rev.
21. Nieth A, Verseux C, Romer W. 2014. Springer Sci. Rev. 3(1):1-11
22. Duerkop B A, Hooper L V. 2013. Nat. Immunol. 14(7):654-59
23. Hsia R. Ohayon H, Gounon P, Dautry-Varsat A, Bavoil P M. 2000. Microbes Infect. 2(7):761-72
24. Merril C R, Biswas B. Carlton R. Jensen N C, Creed G J, et al. 1996. Proc. Natl. Acad. Sci. 93(8):3188-92
25. Tao P, Mahalingam M, Marasa B S, Zhang Z, Chopra A K, Rao V B. 2013. Proc. Nal. Acad. Sci. U.S.A 110(15):5846-51
26. Górski A, D [symbol] browska K. Hodyra-Stefaniak K, Borysowski J, Mi [symbol] dzybrodzki R, Weber-D [symbol] browska B. 2015. Future Microbiol. 10(2):199-204
27. Ivanenkov V V., Menon A G. 2000. Biochem. Biophys. Res. Commun. 276(1):251-57
28. Carrera M R A, Kaufmann G F, Mee J M, Meijler M M, Koob G F, Janda K D. 2004. Proc. Nal. Acad. Sci. U.S.A 101(28):10416-21
29. Aronow R, Danon D, Shahar A, Aronson M. 1964. J. Exp. Med. 120(5):
30. Tian Y, Wu M, Liu X, Liu Z, Zhou Q, et al. 2015. Adv. Healthc. Mater. 4(3):413-19
31. Zhang L, Sun L, Wei R, Gao Q, He T et al. 2016. Antimicrob. Agents Chemother. AAC.01990-16
32. Raetz C R H, Reynolds C M, Trent M S, Bishop R E. 2007. Annu. Rev. Biochem. 76:295-329
33. Morrison D C, Ulevitch R J. 1978. Am. J. Pathol. 93(2):526-618
34. Bonilla N. Rojas M I. Netto Flores Cruz G, Hung S-H, Rohwer F. Barr J J. 2016. PeerJ. 4:e2261
35. Klausner R D, Donaldson J G, Lippincott-Schwartz J. 1992. J. Cell Biol. 116(5):1071-80
36. Huber L A, Pfaller K, Vietor I. 2003. Circ. Res. 92(9):
37. Haynes M, Rohwer F. 2011. In Metagenomics of the Human Body, pp. 63-77. New York, N.Y.: Springer New York
38. Savage D C. 1977. Annu. Rev. Microbiol. 31(1):107-33
39. Sender R, Fuchs S, Milo R, Berg R D, Bianconi E, et al. 2016. Cell. 164(3):337-40
40. Kim M-S, Park E-J, Roh S W, Bae J-W. 2011. Appl. Environ. Microbiol. 77(22):8062-70
41. Reyes A, Wu M, McNulty N P, Rohwer F L, Gordon J I. 2013. Proc. Nal. Acad. Sci. U.S.A 110(50):20236-41
42. Barr, J J, Auro R Furlan M, Whiteson K L, Erb M L, et al. 2013. Proc. Natl. Acad. Sci. 110(26):10771-76
43. Rescigno M, Urbano M, Valzasina B, Francolini M, Rotta G. et al. 2001. Nat. Immunol. 2(4):361-67
44. Jeurink P V, van Bergenhenegouwen J, Jimenez E, Knippels L M J, Femindez L, et al. 2013. Benef. Microbes. 4(1):17-30
45. Dutilh B E, Cassman N, McNair K, Sanchez S E, Silva G G Z, et al. 2014. Nat. Commun.5:
46. Manrique P, Bolduc B, Walk S T, van der Oost J, de Vos W M, Young M J. 2016. Proc. Nal. Acad. Sci. U.S.A 113(37):10400-405
47. Youle M, Haynes M, Rohwer F L. 2012. In Viruses: Essential agents of life, pp. 61-81. Netherlands: Springer
48. Mokili J L, Rohwer F, Dutilh B E. 2012. Curr. Opin. Virol. 2(1):63-77
49. Dabrowska K, Opolski A, Wietrzyk J, Switala-Jelen K, Godlewska J, et al. 2004. Anticancer Res. 24(6):3991-95
50. FemAndez L, Langa S, Martin V, Maldonado A, Jimenez E, et al. 2013. Pharmacol. Res. 69(1):1-10
51. Bloch H. 1940. Arch. Virol. 1:481-96
52. Majewska J, Beta W, Lecion D, Hodyra-Stefaniak K, Klopot A. et al. 2015. Viruses. 7(8):4783-99
53. Sakaguchi S, Yamaguchi T, Nomura T, Ono M. 2008. Cell. 133(5):775-87
54. Belkaid Y, Rouse B T. 2005. Nat. Immunol. 6(4):353-60
55. Bordenstein S R, Bordenstein S R. 2016. Nat. Commun. 7:504-10
56. Grice E A, Segre J A. 2012. Annu. Rev. Genomics Hum. Genet. 13:151-70

### REFERENCES FOR METHODS

1. Bonilla N, Rojas M I, Netto Flores Cruz G, Hung S-H, Rohwer F, Barr J J. 2016. PeerJ. 4:e2261
2. Tesar D B, Cheung E J, Bjorkman P J. 2008. Mol. Biol. Cell. 19(4):1587-93
3. Mu R, Cutting A S, Del Rosario Y, Villarino N, Stewart L, et al. 2016. PLoS One. 11(4):e0153891
4. Weissgerber T L, Milic N M, Winham S J, Garovic V D, Cooper R, et al. 2015. PLOS Biol. 13(4):e1002128
5. Snyder W S, Cook M J, Nasset E S, Karhausen L R, Parry Howells G, Tipton I H. 1975. Report on the Task Group on Reference Man. Pergamon Press: Oxford
6. Sender R, Fuchs S, Milo R, Berg R D, Bianconi E, et al. 2016. Cell. 164(3):337-40
7. Ackermann H-W. 2007. Arch. Virol. 152(2):227-43
8. Release on the IAPWS Formulation 2008 for the Viscosity of Ordinary Water Substance. 2008. http://www.iapws.org/

A number of embodiments of the invention have been described. Nevertheless, it will be understood that various modifications may be made without departing from the spirit and scope of the invention. Accordingly, other embodiments are within the scope of the following claims.

1. A method for:
(a) treating, ameliorating and/or preventing a bacterial or viral infection in an animal in vivo,
wherein optionally the bacterial or viral infection in the animal is inside or outside of the gut of the animal,
wherein optionally the bacterial or viral infection comprises a gut, muscle, lung, liver, kidney or blood or sepsis infection, or a secondary infection inside or outside of the gut,
and optionally the animal is a mammal or a human,
(b) generating or modulating an immune response in an animal wherein optionally the animal is a mammal or a human,
wherein optionally the immune response is a humoral (antibody) response, a cell-mediated immune response, or a tolerogenic immune (suppressing) response, and optionally the modulating of the immune response decreases, ameliorates or inhibits inflammation or an autoimmune reaction in the animal,
and optionally the decreasing, ameliorating or inhibiting of inflammation or the autoimmune reaction in the animal treats, ameliorates, decreases the severity of or inhibits a disease or condition caused by an inflammation or an autoimmune reaction or a disease or condition causing an inflammation or autoimmune reaction,
and optionally the immune response is modulated by inclusion of, release from or display on the surface of (i) the bacteriophage or phage, (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v), an immunogen or a tolerogen, or
(c) treating, ameliorating and/or preventing a disease or condition in an individual in need thereof,
wherein optionally the disease or condition comprises obesity, diabetes, autism, a cystic fibrosis, an inflammation outside or outside of the gut,
and optionally the individual is an animal, a mammal, or a human,
the method comprising:
administering or applying to the animal in need thereof, optionally in vivo, or to the individual in need thereof; or, or administering or applying or inserting into or onto the eukaryotic cell:
(a) (i) a bacteriophage or phage,
(ii) a prophage, a phagemid or a phage-like particle,
(iii) a general transducing agent (GTA), or a small, tailed bacteriophage-like particle,
(iv) a Metamorphosis Associated Contractile structure (MACs),
(v) a phage-derived product, or
(vi) any combination of (i) to (v); or,
(b) a composition, a product of manufacture, a food, a drink, a nutraceutical, a formulation, a pharmaceutical or a pharmaceutical preparation comprising: (i) the bacteriophage or phage, (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v),
wherein optionally the (i) the bacteriophage or phage, (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v) is: chemically or structurally modified, genetically engineered, or is a synthetic version or construct,
and optionally the (i) the bacteriophage or phage, (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v), comprises or has contained thereon or within a payload,
wherein optionally the payload comprises a composition heterologous to (i) the bacteriophage or phage, (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product,
and optionally the heterologous composition is capable of treating, ameliorating and/or preventing a disease or condition in the individual in need thereof, or repairing a defect in the eukarotic cell, or adding or modifying a function in the eukaryotic cell, or altering the genome of or a nucleic acid in the eukaryotic cell, and optionally the (i) the bacteriophage or phage, (ii) the prophage, the phagemid or the phage-like particle, (iii) the general transducing agent (GTA), or the small, tailed bacteriophage-like particle, (iv) the Metamorphosis Associated Contractile structure (MACs), (v) the phage-derived product, or (vi) any combination of (i) to (v), has a size ranging from between about 1 nm and 1000 nm, or between about 100 and 500 nm, or between about 1 nm and 10 μm.

2. The method of claim 1, wherein the individual is a mammal or a human, and optionally the mammal is a human, a human infant, and optionally the animal is wildlife, livestock, beef, poultry, or a domesticated or a laboratory animal.

3. The method of claim 1, wherein an antacid or a buffer or buffering agent or a pharmaceutically acceptable excipient is administered before, during or after, or before and during, administration of the composition, product of manufacture, food, drink, nutraceutical, formulation, pharmaceutical or pharmaceutical preparation, or
a sufficient amount of antacid, buffer or buffering agent is administered (optionally administered before, during or after, or before and during, administration of the composition, product of manufacture, food, drink, nutraceutical, formulation, pharmaceutical or pharmaceutical preparation) to raise the pH of the stomach in the individual to between about 2.5 and 7, or between about 3 and 6.5, or to about 5.0, 5.5, 6.0, 6.5, 6.8 or 7.0, and optionally these pH values reached before, during or after, or before and during, administration,
and optionally the buffer or a buffering agent or the pharmaceutically acceptable excipient comprises an inorganic salt, a citric acid, a sodium chloride, a potassium chloride, a sodium sulfate, a potassium nitrate, a sodium phosphate monobasic, a sodium phosphate dibasic or combinations thereof,
and optionally the antacid comprises a calcium carbonate, a magnesium hydroxide, a magnesium oxide, a magnesium carbonate, an aluminum hydroxide, a sodium bicarbonate or a dihydroxyaluminum sodium carbonate.

4. The method of claim 1, the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is capable of specifically binding to an animal cell (optionally a mammalian or a human cell), or is capable of specifically binding to a specific animal cell,
and optionally the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is engineered to target a specific cell, tissue or organ, or diseased, infected or abnormal cell.

5. The method of claim 1, wherein an immune response is generated by:
a display of epitopes or immunogens, or tolerogens, or immune response modulators, on the surface of the delivered or administered: (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), or
an expression or inclusion of epitopes or immunogens, or tolerogens, or immune response modulators in the delivered or administered: (i) bacteriophage (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v).

6. The method of claim 1, wherein the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is formulated per dose, or per serving, or per unit dosage at, or at a total daily dose of: between about 10(1) (or 101) and 10(20) plaque-forming units (PFUs), or between about 10(3) and 10(17) PFUs, or between about 10(5) and 10(12) PFUs, or between about 10(7) and 10(9) PFUs.

7. The method of claim 1, wherein the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), comprises, or contains within or upon, or carries, a payload or a composition,
wherein optionally the composition or the payload comprises:
a drug;
a modulator of transcytosis, endocytosis, exocytosis, receptor mediated endocytosis, non-specific binding, pinocytosis or macrocytosis in a eukaryotic cell;
an immune response modulator, an epitope, an immunogen or a tolerogen; an antibiotic or a bacteriostatic agent;
a cytotoxic agent;
a nucleic acid (optionally an RNA (optionally an iRNA or miRNA), or an antisense nucleic acid, or a ribozyme, or a CRISPR or CRISPR/Cas9 nucleic acid, or a CRISPR/Cas9-gRNA complex for genome editing, or a DNA), wherein optionally the nucleic acid is derived from a phage, a bacterial or an animal, and optionally the nucleic acid is a synthetic or a recombinantly engineered nucleic acid,
optionally the nucleic acid comprises a eukaryotic gene with the appropriate regulatory motifs, optionally promoters, such that the gene is expressed in a eukaryotic cell, optionally a gut cell;
a genome or fragment thereof, wherein optionally the genome is derived from a phage or a bacterial genome;
a carbohydrate, a protein or peptide, a lipid, an antibody or a small molecule;
a label or tag or a fluorescent molecule or a radiopaque molecule;
a magnetic particle;
a radionucleotide;
a carbohydrate binding domain (CBD) or a moiety or domain capable of binding to: a protein or peptide, a nucleic acid (optionally an RNA or a DNA), a lipid, a lipopolysaccharide or a mucopolysaccharide; or, any combination thereof,
wherein optionally the modulator of transcytosis, endocytosis, exocytosis, receptor mediated endocytosis, non-specific binding, pinocytosis or macrocytosis in the eukaryotic cell comprises or is an inhibitor or enhancer of transcytosis, endocytosis, exocytosis, receptor mediated endocytosis, non-specific binding, pinocytosis or macrocytosis, and optionally the inhibitor of transcytosis, endocytosis, exocytosis, receptor mediated endocytosis, non-specific binding, pinocytosis or macrocytosis is or comprises N-ethylmaleimide (NEM), chlorpromazine, filipin, colchicine, dynasore, Concanamycin C (Con C), eeyarestatin I, Golgicide A, Leptom cin B, levetiracetam, or brefeldin A (BFA), or an antibody that inhibits PIKFyve or a SNARE protein or an antibody that blocks SNARE assembly,
and optionally the nucleic acid is or comprises a small inhibitory RNA (siRNA), an antisense nucleic acid or RNA, or a CRISPR nucleic acid or
CRISPR/Cas9 system comprising a synthetic guide RNA (gRNA) and/or a nuclease, or the nucleic acid encodes a protein or a small inhibitory RNA (siRNA), an antisense RNA, or a CRISPR nucleic acid or CRISPR/Cas9 system comprising a synthetic guide RNA (gRNA) and or a nuclease,
and optionally the nucleic acid is contained in an expression vehicle or vector, and optionally the nucleic acid is operatively linked to a transcriptional control motif, which optionally can be a promoter and/or enhancer, optionally a tissue or cell specific, or constitutive, or inducible, promoter and/or enhancer,
and optionally the payload or composition is delivered to or released in, onto or into the eukaryotic cell, or is delivered or released into a eukaryotic cell subcellular compartment or an organelle,
and optionally the eukaryotic cell subcellular compartment or organelle is a cytoplasm, an endosome, an exosome, a liposome, a nucleus, a nucleosome, a golgi, an endoplasmic reticulum (ER) or a mitochondrion,
and optionally the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is engineered to release the payload or composition into the eukaryotic cell, or eukaryotic cell subcellular compartment or organelle, or into a specific eukaryotic cell subcellular compartment or organelle,
and optionally the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is degraded in a lysosome, or is engineered or designed to be degraded in a lysosome.

8. The method of claim 1, wherein the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is or is derived from, or is substantially or partially derived from:
(a) a prokaryotic bacteriophage, optionally a bacterial or an Archaeal bacteriophage;
(b) a prokaryotic bacteriophage of the order Caudovirales or Ligamenvirales;
(c) a prokaryotic bacteriophage of the family Myoviridae, Siphoviridae, Podoviridae, Lipothrixvihdae, Rudiviridae, Ampullaviridae, Bicaudaviridae, Clavaviridae, Corticoviridae, Cystoviridae, Fuselloviridae, Globuloviridae, Gutta viridae, Inoviridae, Leviviridae, Microviridae, Plasmaviridae or Tectivirus or a combination thereof;
(d) a Bacteroidetes-infecting phage or a class 1 filamentous phage, or an F1 or an Fd filamentous bacteriophage;
(e) a bacteriophage QP virus-like particle; or
(f) an Enterobacteria phage T4, a lambda phage, an M13 Inoviridae phage, a crAss phage, or a phage capable of infecting a mammalian or a human gut.

9. The method of claim 1, wherein the (i) bacteriophage or phage, (ii) prophage, the phagemid or phage-like particle, (iii) general transducing agent (GTA), or small, tailed bacteriophage-like particle, (iv) Metamorphosis Associated Contractile structure (MACs), (v) phage-derived product, or (vi) any combination of (i) to (v), is a chemically or structurally modified bacteriophage, phagemid or phage-like particle,
and optionally the exterior (outer) surface of bacteriophage, phagemid or phage-like particle, comprises:
(a) at least one heterologous:
(i) carbohydrate binding domain (CBD),
(ii) a moiety or domain capable of binding to a component of a mucus, optionally a mucus of or derived from: a mammalian mucus membrane, a gut, a urinary, a reproductive, an animal or an environmental mucus,
optionally capable of binding to a mucus or mucus-like macromolecule, a mucin, a fatty acid, a phospholipid, a cholesterol, an elastin, a glycoprotein, a mucin glycoprotein or glycan, a mucin protein, a humic acid, a cellulose, a chitin, a high molecular weight (MW) polysaccharide, an N-acetylgalactosamine, an N-acetylglucosamine, a fucose, a galactose, a sialic acid (N-acetylneuraminic acid) a mannose, or any combination thereof,
and optionally the moiety or domain capable of binding to a component of a mucus directs or targets the bacteriophage, phagemid or phage-like particle to a specific region of a mucosal surface that overlaps with a bacterial host range, and optionally the specific region comprises a mucosal surface basal layer, a mucosal surface apical layer, a mucosal surface lumen, a mucus layer, or a mucosal surface having a concentration of between about 0% to 1% mucin, or between about 1% to 5%, or a mucin concentration of between about 1% to 10%,
and optionally the moiety or domain capable of binding to a component of a mucus directs or that targets the bacteriophage, phagemid or phage-like particle to a specific region of a mucosal surface allows the bacteriophage, phagemid or phage-like particle to reside or concentrate or persist in a specific region of the mucosal surface that overlaps with a bacterial host range,
and optionally the bacteriophage, phagemid or phage-like particle is adapted to a physico-chemical environment of the mucus or specific
region of a mucosal surface, and the physico-chemical environment optionally comprises: a pH range of between about 6 to 8, a pH range of between about 4 to 10, a pH range of between about 1 to 12, an ionic concentration of between about 1 mg to 1000 mg, an ionic concentration of between about 1 μg to 1000 g, an ionic concentration of between about 1 pgm to 1000 kg, a temperature change of between about 35° C. to 42° C., a temperature change of between about 25° C. to 55° C., or a temperature change of between about 1° C. to 99° C.;
(iii) moiety or domain capable of binding to a protein or peptide, a protein or peptide (optionally an antibody or antigen binding fragment thereof, an antigen, an immunogen, a tolerogen), a glycoprotein, a nucleic acid (optionally an RNA or a DNA), a lipid or cholesterol, a lipopolysaccharide, a mucopolysaccharide, a gel, a hydrogel, a complex fluid, or a combination thereof, or
(iv) combination of any of (i) to (iii),
wherein optionally the heterologous CBD is a bacteriophage carbohydrate binding domain (CBD), and optionally the heterologous CBD is a CBD derived from a different species, genus, family or order of bacteriophage; or the CBD is a mammalian or a human CBD,
and optionally any of (i) to (iii) comprises or has structural homology to: a C-type lectin, a lectin, a bacteriodetes-associated carbohydrate-binding often N-terminal (BACON) domain, a Brefeldin A-inhibited guanine nucleotide-exchange factor for ADP-ribosylation factor (Big, optionally Big1, Big2, or Big3), a polycystic kidney disease domain (PKD), a Fibronectin type 3 homology domain (Fn3), a Hyalin Repeat (HYR) domain, an Ig_2 domain, an immunoglobulin I-set domain, a carbohydrate-adherence domain, a mucus-binding protein, a glycan-binding protein, a protein-binding protein, a mucus-adhering protein or a mucus-adhering glycoprotein;
b) additional homologous CBDs (more CBDs than found on a comparable wild type (WT) bacteriophage); or
(c) a combination of (a) and (b).

10. The method of claim 1, wherein the
(i) a bacteriophage or phage, wherein optionally the phage is a temperate phage or a lysogenic phage,
(ii) a prophage, wherein optionally the prophage is a tailocin, or a defective prophage where head and tail are absent but the prophage is otherwise adsorption-competent, a phagemid or a phage-like particle, wherein optionally the phagemid or a phage-like particle is a phagocin),
(iii) a general transducing agent (GTA), or a small, tailed bacteriophage-like particle,
(iv) a Metamorphosis Associated Contractile structure (MACs),
(v) a phage-derived product (optionally an endolysin, aholin, a lysoz me, or a tail fiber protein), or
(vi) any combination of (i) to (v),
comprises or has contained therein a genome (optionally a substantially complete or a partial, or a genetically engineered or hybrid genome) that is altered such that after reproduction in a host cell (optionally a bacterial host cell), or in an in vitro system, the exterior (outer) surface of the bacteriophage comprises:
(a) at least one non-bacteriophage carbohydrate binding domain (CBD), and optionally the CBD is a mammalian or a human CBD;
(b) at least one heterologous bacteriophage CBD, wherein optionally the heterologous CBD is a CBD from a different species, genus, family or order of bacteriophage;
(c) more CBDs than found on a wild type (WT) (comparable) bacteriophage; or
(d) at least one moiety or domain capable of binding to a component of a mucus,
optionally a mucus of or derived from: a mammalian mucus membrane, a gut, a urinary, a reproductive, an animal environmental mucus,
optionally capable of binding to a mucus or mucus-like macromolecule, a mucin, a fatty acid, a phospholipid, a cholesterol, an elastin, a glycoprotein, a mucin glycoprotein or glycan, a mucin protein, a humic acid, a cellulose, a chitin, a high molecular weight (MW) polysaccharide, an N-acetylgalactosamine, an N-acetylglucosamine, a fucose, a galactose, a sialic acid (N-acetylneuraminic acid) a mannose, or any combination thereof,
and optionally the moiety or domain capable of binding to a component of a mucus directs or targets the phage to a specific region of a mucosal surface that overlaps with a bacterial host range, and optionally the specific region comprises a mucosal surface basal layer, a mucosal surface apical layer, a mucosal surface lumen, a mucus layer, or a mucosal surface having a concentration of between about 0% to 1% mucin, or between about 1% to 5%, or a mucin concentration of between about 1% to 10%,
and optionally the moiety or domain capable of binding to a component of a mucus directs or that targets the phage to a specific region of a mucosal surface allows the phage to reside or concentrate or persist in a specific region of the mucosal surface that overlaps with a bacterial host range,
and optionally the phage is adapted to a physico-chemical environment of the mucus or specific region of a mucosal surface, and the physico-chemical environment optionally comprises: a pH range of between about 6 to 8, a pH range of between about 4 to 10, a pH range of between about 1 to 12, an ionic concentration of between about 1 mg to 1000 mg, an ionic concentration of between about 1 μg to 1000 gram (g), an ionic concentration of between about 1 pgm (picogram) to
1000 kg, a temperature change of between about 35° C. to 42° C., a temperature change of between about 25° C. to 55° C., or a temperature change of between about 1° C. to 99° C.;
(e) at least one moiety or domain capable of binding to a protein or peptide, a protein or peptide (optionally an antibody or antigen binding fragment thereof, an antigen, an immunogen, a tolerogen), a glycoprotein, a nucleic acid (optionally an RNA or a DNA), a lipid or cholesterol, a lipopolysaccharide, a mucopolysaccharide, a gel, a hydrogel, a complex fluid, or a combination thereof; or
(f) any combination of (a) to (e),
and optionally any of (a) to (e) comprises or has structural homology to: a C-type lectin, a lectin, a bacteriodetes-associated carbohydrate-binding often N-terminal (BACON) domain, a Brefeldin A-inhibited guanine nucleotide-exchange factor for ADP-ribosylation factor (Big, optionally Big1, Big2, or Big3), a polycystic kidney disease domain (PKD), a Fibronectin type 3 homology domain (Fn3), a Hyalin Repeat (HYR) domain, an Ig-2 domain, an immunoglobulin I-set domain, a carbohydrate-adherence domain, a mucus-binding protein, a glycan-binding protein, a protein-binding protein, a mucus-adhering protein or a mucus-adhering glycoprotein.

11. The method claim 9, wherein:
(a) the CBD is entirely, or substantially, a synthetic or non-natural CBD, optionally an antibody or antigen binding domain that specifically binds to a carbohydrate;
(b) the CBD is or comprises a protein having a carbohydrate-binding-like fold, which optionally comprises a seven-stranded beta-sandwich, or optionally is or
comprises an immunoglobulin-like binding domain, or a protein domain comprising a 2-layer sandwich of between 7 and 9 antiparallel I2-strands arranged in two P-sheets;
(c) the CBD is or is derived from or has substantial structural identity (homology) to a mammalian or a human CBD;
(d) the bacteriophage is known or demonstrated to be toxic or lysogenic to a bacteria, or the bacteriophage is bactericidal or bacteriostatic, or the bacteriophage can treat, inhibit or prevent an infection, and optionally the bacteriophage is engineered to specifically bind to or target the bacteria,
wherein optionally the bacteriophages are bactericidal or bacteriostatic to a gram-negative bacterium or a gram-positive bacterium, and optionally the bacteriophage is engineered to specifically bind to or target the gram-negative bacteria or gram-positive bacteria,
and optionally the bacteria or infection is or is caused by an MSRA infection, a Staphylococcus, a Staphylococcus aureus, a Clostridium, a Clostridium difficile, an Escherichia coli, a. Shigella, a. Salmonella, a. Campylobacter, a Chloerae, a Bacillus, a Yersinia or a combination thereof, and optionally the bacteriophage is engineered to specifically bind to or target the bacteria or
(e) the bacteriophage is made or identified by a process comprising: screening a plurality of bacteriophages for bactericidal or bacteriostatic properties against a bacteria of interest, and selecting the bacteriophages having a lysogenic or a bactericidal or bacteriostatic activity.

12. The method of claim 9, wherein the CBD is, or is derived from, or has substantial structural identity or homology to:
(a) a protein having a carbohydrate-binding-like fold, which optionally comprises a seven-stranded beta-sandwich, or optionally is or comprises an immunoglobulin-like binding domain, or comprises a protein domain comprising a 2-layer sandwich of between 7 and 9 antiparallel F-strands arranged in two P-sheets;
(b) a CBD, optionally an antibody or antigen binding fragment thereof, capable of specifically binding to a tumor associated carbohydrate antigen (TACA); or
(c) a carbohydrate-binding module family 1 (CBM1);
a carbohydrate-binding module family 2 (CBM2);
a carbohydrate-binding module family 3 (CBM3);
a carbohydrate-binding module family 4 (CBM4);
a carbohydrate-binding module family 5 (CBM5);
a carbohydrate-binding module family 6 (CBM6);
a carbohydrate-binding module family 7 (CBM7);
a carbohydrate-binding module family 8 (CBM8);
a carbohydrate-binding module family 9 (CBM9);
a carbohydrate-binding module family 10 (CBM10);
a carbohydrate-binding module family 11 (CBM11);
a carbohydrate-binding module family 12 (CBM12);
a carbohydrate-binding module family 13 (CBM13);
a carbohydrate-binding module family 14 (CBM14);
a carbohydrate-binding module family 15 (CBM15);
a carbohydrate-binding module family 16 (CBM16);
a carbohydrate-binding module family 17 (CBM17);
a carbohydrate-binding module family 18 (CBM18);
a carbohydrate-binding module family 19 (CBM19);
a carbohydrate-binding module family 20 (CBM20);
a carbohydrate-binding module family 21 (CBM21);
a carbohydrate-binding module family 25 (CBM25);
a carbohydrate-binding module family 27 (CBM27);
a carbohydrate-binding module family 28 (CBM28);
a carbohydrate-binding module family 33 (CBM33);
a carbohydrate-binding module family 48 (CBM48); or,
a carbohydrate-binding module family 49 (CBM49).

13. (canceled)

14. A therapeutic formulation comprising
(a)(i) a bacteriophage or phage, wherein optionally the phage is a temperate phage or a lysogenic phage,
(ii) a prophage, wherein optionally the prophage is a tailocin, or a defective prophage where head and tail are absent but the prophage is otherwise adsorption-competent, a phagemid or a phage-like particle, wherein optionally the phagemid or a phage-like particle is a phagocin),
(iii) a general transducing agent (GTA), or a small, tailed bacteriophage-like particle,
(iv) a Metamorphosis Associated Contractile structure (MACs),
(v) a phage-derived product (optionally an endolysin, aholin, a lysoz me, or a tail fiber protein), or
(vi) any combination of (i) to (v), and
(b) a payload,
wherein optionally the payload comprises
a drug;
a modulator of transcytosis, endocytosis, exocytosis, receptor mediated endocytosis, non-specific binding, pinocytosis or macrocytosis in a eukaryotic cell;
an immune response modulator, an epitope, an immunogen or atolerogen; an antibiotic or a bacteriostatic agent;
a cytotoxic agent;
a nucleic acid,
wherein optionally the nucleic acid comprises:
an RNA (optionally an iRNA or miRNA), or an antisense nucleic acid, or a ribozyme, or a CRISPR or CRISPR/Cas9 nucleic acid, or a CRISPR/Cas9-gRNA complex for genome editing, or a DNA), wherein optionally the nucleic acid is derived from a phage, a bacterial or an animal, and optionally the nucleic acid is a synthetic or a recombinant-}′ engineered nucleic acid,
a eukaryotic gene, optionally comprising regulatory motifs, optionally promoters, such that the gene is expressed in a eukaryotic cell, optionally a gut cell;
a small inhibitory RNA (siRNA), an antisense nucleic acid or RNA, or a CRISPR nucleic acid or CRISPR/Cas9 system comprising a synthetic guide RNA (gRNA) and/or a nuclease, or the nucleic acid encodes a protein or a small inhibitory RNA (siRNA), an antisense RNA, or a CRISPR nucleic acid or CRISPR/Cas9 system comprising a synthetic guide RNA (gRNA) and or a nuclease,
a genome or fragment thereof, wherein optionally the genome is derived from a phage or a bacterial genome,
and optionally the nucleic acid is contained in an expression vehicle or vector, and optionally the nucleic acid is operatively linked to a transcriptional control motif, which optionally can be a promoter and/or enhancer, optionally a tissue or cell specific, or constitutive, or inducible, promoter and/or enhancer;
a carbohydrate, a protein or peptide, a lipid, an antibody or a small molecule; a label or tag or a fluorescent molecule or a radiopaque molecule;
a magnetic particle;
a radionucleotide;
a carbohydrate binding domain (CBD) or a moiety or domain capable of binding to: a protein or peptide, a nucleic acid (optionally an RNA or a DNA), a lipid, a lipopolysaccharide or a mucopolysaccharide; or, any combination thereof,
wherein optionally the modulator of transcytosis, endocytosis, exocytosis, receptor mediated endocytosis, non-specific binding, pinocytosis or macrocytosis in the eukaryotic cell comprises or is an inhibitor or enhancer of transcytosis, endocytosis, exocytosis, receptor mediated endocytosis, non-specific binding, pinocytosis or macrocytosis, and optionally the inhibitor of transcytosis, endocytosis, exocytosis, receptor mediated endocytosis, non-specific binding, pinocytosis or macrocytosis is or comprises N-ethylmaleimide (NEM), chlorpromazine, filipin, colchicine, dynasore, Concanamycin C (Con C), eeyarestatin I, Golgicide A, Leptom cin B, levetiracetam, or brefeldin A (BFA), or an antibody that inhibits PIKFyve or a SNARE protein or an antibody that blocks SNARE assembly.
